(19) United States
(12) Patent Application Publication
(10) Pub. No.: US 2022/0267323 A1
(43) Pub. Date: Aug. 25, 2022

(54) SYNTHESIS OF BICYCLIC INHIBITORS OF HISTONE DEACETYLASE

(71) Applicant: Alkermes, Inc., Waltham, MA (US)

(72) Inventors: Nathan Oliver Fuller, Arlington, MA (US); Michael Charles Hewitt, Brookline, MA (US); Sylvie M. Asselin, Broomfield, CO (US); Wayne Douglas Luke, West Lafayette, IN (US)

(21) Appl. No.: 17/628,049

(22) PCT Filed: Jul. 23, 2020
(86) PCT No.: PCT/US2020/043177

§ 371 Date: Jan. 18, 2022

Related U.S. Application Data
(60) Provisional application No. 62/877,363, filed on Jul. 23, 2019.

(51) Int. Cl. C07D 471/04; C07D 409/04; C07D 487/04

(52) U.S. Cl. CPC C07D 471/04; C07D 487/04; C07D 409/04

(57) ABSTRACT

Provided herein are synthetic methods for the preparation of compounds having the Formula: (I) and (I′) and salts thereof.

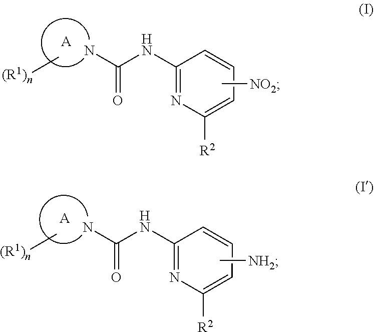

### RELATED APPLICATIONS

This application claims priority to U.S. Provisional Application No. 62/877,363, filed Jul. 23, 2019, the entire contents of which are incorporated by reference herein.

### BACKGROUND

Inhibitors of histone deacetylases (HDAC) have been shown to modulate transcription and to induce cell growth arrest, differentiation and apoptosis. HDAC inhibitors also enhance the cytotoxic effects of therapeutic agents used in cancer treatment, including radiation and chemotherapeutic drugs. Marks, P., Rifkind, R. A., Richon, V. M., Breslow, R., Miller, T., Kelly, W. K. Histone deacetylases and cancer: causes and therapies. Nat Rev Cancer, 1, 194-202, (2001); and Marks, P. A., Richon, V. M., Miller, T., Kelly, W. K. Histone deacetylase inhibitors. Adv Cancer Res, 91, 137-168, (2004). Moreover, recent evidence indicates that transcriptional dysregulation may contribute to the molecular pathogenesis of certain neurodegenerative disorders, such as Huntington's disease, spinal muscular atrophy, amyotropic lateral sclerosis, and ischemia. Langley, B., Gensert, J. M., Beal, M. F., Ratan, R. R. Remodeling chromatin and stress resistance in the central nervous system: histone deacetylase inhibitors as novel and broadly effective neuroprotective agents. Curr Drug Targets CNS Neurol Disord, 4, 41-50, (2005). A recent review has summarized the evidence that aberrant histone acetyltransferase (HAT) and histone deacetylases (HDAC) activity may represent a common underlying mechanism contributing to neurodegeneration. Additionally, using a mouse model of depression, Nestler has recently highlighted the therapeutic potential of histone deacetylation inhibitors (HDAC5) in depression. Tsankova, N. M., Berton, O., Renthal, W., Kumar, A., Neve, R. L., Nestler, E. J. Sustained hippocampal chromatin regulation in a mouse model of depression and antidepressant action. Nat Neurosci, 9, 519-525, (2006).

The role of individual HDACs in long-term memory has been explored in two recent studies. Kilgore et al. 2010, Neuropsychopharmacology 35:870-880 revealed that nonspecific HDAC inhibitors, such as sodium butyrate, inhibit class I HDACs (HDAC1, HDAC2, HDAC3, HDAC8) with little effect on the class IIa HDAC family members (HDAC4, HDAC5, HDAC7, HDAC9). This suggests that inhibition of class I HDACs may be critical for the enhancement of cognition observed in many studies. Indeed, forebrain and neuron specific overexpression of HDAC2, but not HDAC1, decreased dendritic spine density, synaptic density, synaptic plasticity and memory formation. (Guan et al., 2009, Nature, 459:55-60). In contrast, HDAC2 knockout mice exhibited increased synaptic density, increased synaptic plasticity and increased dendritic density in neurons. These HDAC2 deficient mice also exhibited enhanced learning and memory in a battery of learning behavioral paradigms. This work demonstrates that HDAC2 is a key regulator of synaptogenesis and synaptic plasticity. Additionally, Guan et al. showed that chronic treatment of mice with SAHA (an HDAC 1, 2, 3, 6, 8 inhibitor) reproduced the effects seen in the HDAC2 deficient mice and rescued the cognitive impairment in the HDAC2 overexpressing mice.

Effective HDAC inhibitors are disclosed in WO 2017/007756 and WO 2018/132531, the contents of which are incorporated herein by reference. Of particular interest are Compound 1 and Compound 5, exemplified in U.S. Pat. No. 9,951,069, the contents of which are incorporated herein by reference, having the structures:

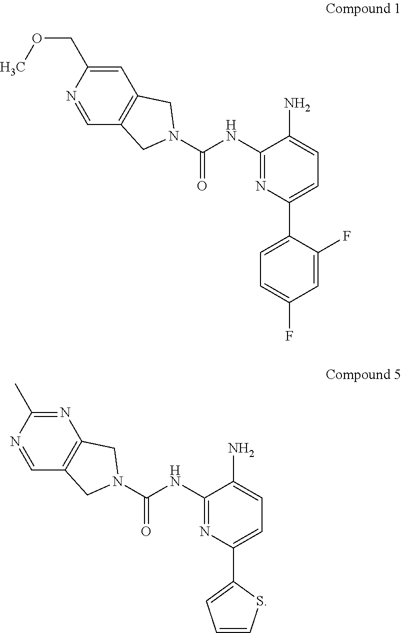 [SMILES: COCc1cc2c(cn1)CN(C(=O)Nc1nc(-c3ccc(F)cc3F)ccc1N)C2 ; Cc1ncc2c(n1)CN(C(=O)Nc1nc(-c3cccs3)ccc1N)C2]

Compound 1 and Compound 5 are potent and selective small molecule inhibitors of the HDAC-CoREST complex. Preclinical data for Compound 1 and Compound 5 shows advantages in hematological safety, ADME and PK. See e.g., U.S. Pat. No. 9,951,069. Additional preclinical data in animal models suggests that Compound 1 and Compound 5 have extended efficacy and safety.

Compound 1, Compound 5, and related pharmacophores, were synthesized in U.S. Pat. No. 9,951,069, WO 2017/007756, and WO 2018/132531 from the corresponding bis-phenylcarbamate and amine to form the requisite urea as shown below.

General Synthesis

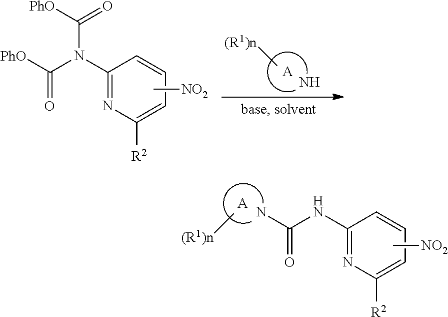

Compound 1 NO₂ Precursor Formation

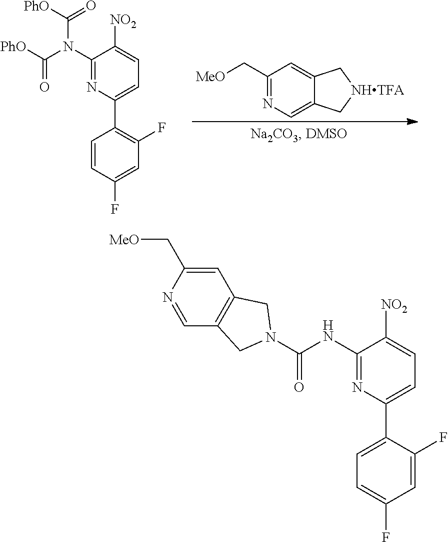

Potential liabilities of this approach are that it requires isolation of the biscarbamate intermediate, and requires at least two equivalents of phenyl carbonchloridate to form the biscarbamate intermediate. This results in the formation of two equivalents of byproduct phenol. Given the therapeutic benefits associated with HDAC inhibitors such as Compound 1 and Compound 5, more efficient and scalable routes are needed to further pharmaceutical process and manufacturing.

### SUMMARY

Disclosed is an improved process for the preparation of a compound having Formula I:

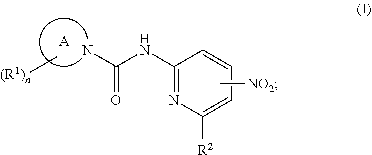

or a salt thereof, wherein R¹, n, A, and R² are as described herein. This process proceeds via the reaction of an isocyanate having Formula II:

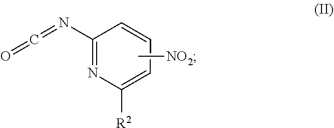

with an amino compound having Formula III:

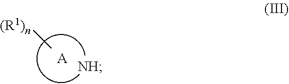

or a salt thereof, to form the compounds of Formula I. R¹, n, A, and R² are as described herein. This process allows for in situ generation of isocyanate intermediates of formula II which allows for the urea formation of compounds of formula I in the same reaction vessel, saving a step from the original approach in an efficient manufacturing process more applicable to larger scale production. The process is also more atom economical, since it does not require two equivalents of phenyl carbon chloridate, thus eliminating two equivalents of phenol being generated as a reaction byproduct.

Also disclosed are improved reduction processes for converting the precursor NO₂ compound of Formula I, or a salt thereof, to the corresponding amine end product. Such process include e.g., reacting a compound having Formula I:

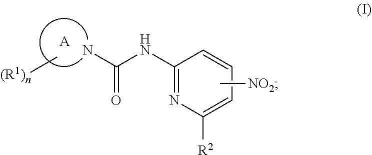

or a salt thereof, with formic acid, an organic base, a metal catalyst and organic solvent, and optionally under an atmosphere of hydrogen to form a compound of Formula I′:

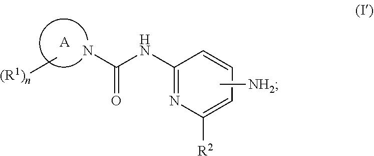

wherein R¹, n, A, and R² are as described herein. This process allows for more efficient manufacturing of compounds of Formula I′ on large scale.

The above preparations can be performed on scale (>5 kg) using commercially available reagents.

Further provided are isocyanate compounds having the Formula VIII:

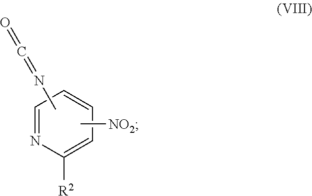

and salts thereof, wherein R² is as described herein.

### BRIEF DESCRIPTION OF THE FIGURES

FIG. 1 depicts an x-ray powder diffraction (XRPD) pattern for the malate salt crystalline Form A of Compound 1 as described in the exemplification section.

FIG. 2 depicts the combined thermogravimetric analysis (TGA) thermogram and differential scanning calorimetry (DSC) thermogram for the malate salt crystalline Form A of Compound 1 as described in the exemplification section.

### DETAILED DESCRIPTION

### A. Definitions

The term “alkyl” when used alone or as part of a larger moiety, such as “haloalkyl”, means a saturated straight-chain or branched monovalent hydrocarbon radical. Unless otherwise specified, an alkyl group typically has 1-4 carbon atoms, i.e., (C₁-C₄)alkyl. For example, “(C₁-C₄)alkyl” includes methyl, ethyl, propyl (e.g., n-propyl or isopropyl) and butyl (e.g., n-butyl, isobutyl, 1-methylpropyl, or tert-butyl).

As used herein, the term “halogen” or “halo” means F, Cl, Br, or I.

The term “haloalkyl” includes mono, poly, and perhaloalkyl groups where the halogens are independently selected from fluorine, chlorine, bromine, and iodine.

The term “hydroxyl” or “hydroxy” refers to —OH.

“Alkoxy” means an alkyl radical attached through an oxygen linking atom, represented by —O-alkyl. For example, “(C₁-C₄)alkoxy” includes methoxy, ethoxy, propoxy, and butoxy.

“Haloalkoxy” is a haloalkyl group which is attached to another moiety via an oxygen atom such as, e.g., but are not limited to —OCHF₂, —OCF₃, and —OCH₂CF₃.

The term “4- to 6-membered monocyclic heteroaryl” or “5- to 6-membered monocyclic heteroaryl” refers to a 4- to 6-membered or 5- to 6-membered monocyclic aromatic radical containing 1-4 heteroatoms selected from N, O, and S. Nonlimiting examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl and pyrazinyl. It will be understood that when specified, optional substituents on a heteroaryl group may be present on any substitutable position and, include, e.g., the position at which the heteroaryl is attached.

The term “heterocyclyl” means a 4- to 12-membered (e.g., a 4- to 7-membered or 4- to 6-membered) saturated or partially unsaturated heterocyclic ring containing 1 to 4 heteroatoms independently selected from N, O, and S. It can be mononcyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridinyl, indolinonyl, dihydropyrrolotriazolyl, dihydropyrrolopyridyl, dihydropyrrolopyrimidinyl, imidazopyrimidinyl, quinolinonyl, tetrahydropyrrolothiazolyl, tetrahydropyrrolopyrazolyl, dioxaspirodecanyl. It will also be understood that when specified, optional substituents on a heterocyclyl group may be present on any substitutable position and, include, e.g., the position at which the heterocyclyl is attached (e.g., in the case of an optionally substituted heterocyclyl or heterocyclyl which is optionally substituted).

The term “spiro” refers to two rings that share one ring atom (e.g., carbon).

The term “fused” refers to two rings that share two adjacent ring atoms with one another.

The term “bridged” refers to two rings that share three ring atoms with one another.

An “isocyanate former” is a substance or a combination of substances that reacts with an amine to form the group “O═C═N—”. The isocyanate former can be phosgene, diphosgene, or triphosgene, carbonyldiimidazole, or a combination of reactants, such as CO₂/Mitsunobu zwitterions or (Boc)₂O/DMAP.

The term “solvent” as used herein refers to an individual solvent or to a mixture of solvents. Solvents may be protic, aprotic, etc. For instance, an aprotic organic solvent or an aprotic solvent, as defined below, could be toluene, or it could be a mixture of toluene and another aprotic solvent such as DMF. Thus, as used herein the term aprotic organic solvent or aprotic solvent could also encompass a toluene/DMF mixture as long as the resulting properties of the mixture are those of an aprotic solvent. The terms “aprotic solvent” and “aprotic organic solvent” are used interchangeably.

Examples of protic solvents include water, alcohols (e.g., methanol, ethanol, propanol, butanol, isopropanol, isobutanol, etc.), formic acid, hydrogen fluoride, nitromethane, acetic acid and ammonia.

Aprotic solvents are usually classified as either polar aprotic or non-polar (or apolar) aprotic depending on the values of their dielectric constants. Apolar or non-polar aprotic solvents usually have small dielectric constants. Examples of polar aprotic solvents include acetonitrile (ACN), anisole, N,N-dimethylformamide (DMF), N,N-dimethylacetamide (DMA), N-methylpyrrolidone (NMP), hexamethylphosporamide (HMPA), tetrahydrofuran, ethyl acetate, acetone, and dimethylsulfoxide (DMSO). Examples of apolar or non-polar aprotic solvents include hexane, pentane, decane and other alkanes, benzene, toluene, 1, 4-dioxane, chloroform, ethers (such as diethyl ether and methyl-tert-butyl ether), dichloromethane and dichloroethane.

As used herein, the term “base” refers to a chemical species that donates electrons, accepts protons, or releases hydroxide (OH—) ions in aqueous solution. Bases include, e.g., organic and inorganic bases. Organic bases include e.g., pyridine, 4-dimethylaminopyridine, 2,3-lutidine, 2,6-lutidine, imidazole, benzimidazole, histidine, guanidine, a phosphazene base, a hydroxide of a quaternary ammonium cation, piperidine, 2,6-ditertbutylpyridine, 1,4-diazabicyclo[2.2.2]octane, or 1,8-diazabicyclo[5.4.0]undec-7-ene. Alkanamines include e.g., methylamine (MeNH₂), dimethylamine (Me₂NH), trimethylamine (Me₃N), ethylamine (EtNH₂), diethylamine (EtNH₂), triethylamine, N,N-disopropylethylamine, aniline (PhNH₂), 4-methoxyaniline (4-MeOC₆H₄NH₂), N,N-dimethylaniline (PhNMe₂), 3-nitroaniline (3-NO₂—C₆H₄NH₂), 4-nitroaniline (4-NO₂—C₆H₄NH₂), and 4-trifluoromethylaniline (CF₃C₆H₄NH₂). Inorganic bases include e.g., sodium bicarbonate (NaHCO₃), sodium carbonate (Na₂CO₃), potassium carbonate (K₂CO₃), cesium carbonate (Cs₂CO₃), lithium hydroxide (LiOH), sodium hydroxide (NaOH), potassium hydroxide (KOH), rubidium hydroxide (RbOH), cesium hydroxide (CsOH), magnesium hydroxide (Mg(OH)₂), calcium hydroxide (Ca(OH)₂), strontium hydroxide (Sr(OH)₂), and barium hydroxide (Ba(OH)₂).

As used herein, the term “acid” refers to a chemical species that donates protons or hydrogen ions and/or accepts electrons. Acids include organic and inorganic acids. Organic acids include e.g., acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+)-camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, oleic, orotic, oxalic, palmitic, pamoic, propionic, L-pyroglutamic, salicylic, 4-amino-salicylic, sebacic, stearic, succinic, tannic, tartaric (e.g. (+)-L-tartaric), thiocyanic, trifluoroacetic acid, undecylenic and valeric acid. Inorganic acids include e.g., hydrobromic, hydrochloric, hydriodic, nitric, phosphoric, and sulfuric acid.

As used herein, the term “reductive conditions” refers to a chemical reaction condition under which a molecule, atom or ion gains electrons. The reductive conditions comprise catalytic hydrogenation (e.g., Raney nickel/H₂, Pd(C)/H₂, Pt(C)/H₂, Pd[Fe](C)/H₂, Pt[Fe](C)/H₂, Pt[V](C)/H₂, Pd[V](C)/H₂, Pd[Pt](C)/H₂), reaction with hydride donors (e.g., lithium aluminum hydride and boranes), reaction with metals in acidic media (e.g., iron or zinc with acetic acid), reaction with metals (e.g., tin(II) chloride, titanium(III) chloride, samarium, and zinc), and reaction with acids (e.g., formic acid, ammonium formate, and hydroiodic acid) in the presence of a metal catalyst (e.g., Pd(C)).

Salts may be obtained using standard procedures well known in the art, and include basic or acidic salts. Unless stated otherwise, when malate salts are discussed herein (e.g., the malate salt of Compound 1), they include both the ionic salt forms, i.e., where there are charged cation(s) and anion(s) and neutral salt complexes, i.e., a co-crystal. In one aspect, malate salts described herein are ionic. In another aspect, malate salts described herein are co-crystals. The term “co-crystal” (or “cocrystal”) refers to a multicomponent system in which a host active pharmaceutical ingredient (e.g., Compound 1 or Compound 5) and a guest or co-former molecule or molecules (e.g., malic acid) are arranged in the same lattice in a non-ionic manner. The API and co-former molecules e.g., may interact by hydrogen bonding and possibly other non-covalent interactions without ionic interactions and without significant or complete proton exchange occurs between the API molecule and the guest molecule.

Chemical purity refers to extent by which a disclosed compound (e.g., as formed from a disclosed process) is free from materials having different chemical structures. Chemical purity means the weight of the product or desired compound divided by the sum of the weight of the product or desired compound plus materials/impurities having different chemical structures multiplied by 100%, i.e., percent by weight. In one aspect, compounds formed by the disclosed processes have chemical purities of at least 60%, at least 70%, at least 80%, at least 90%, at least 95%, at least 96%, at least 97%, at least 98%, or at least 99% by weight.

### B. Process Embodiments

In a first embodiment, provided herein is a method for preparing a compound of Formula I,

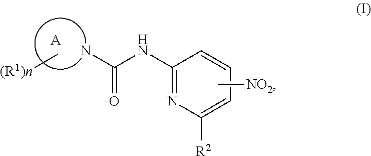

or a salt thereof, wherein:

ring A is a heterocyclyl;

R¹ is halo, (C₁-C₄)alkyl, halo(C₁-C₄)alkyl, (C₁-C₄)alkoxy, halo(C₁-C₄)alkoxy, or 4- to 6-membered monocyclic heteroaryl, where said (C₁-C₄)alkyl is optionally substituted with 1 to 3 groups selected from halo, hydroxyl, (C₁-C₄)alkoxy, —NH(C₁-C₄)alkyl, —N((C₁-C₄)alkyl)₂, and 4- to 6-membered monocyclic heteroaryl, wherein each instance of said 4- to 6-membered monocyclic heteroaryl is optionally substituted with 1 to 2 groups selected from halo, (C₁-C₄)alkoxy, (C₁-C₄)alkyl, and halo(C₁-C₄)alkyl;

n is 0, 1, or 2;

R² is phenyl or 5- to 6-membered monocyclic heteroaryl, each of which is optionally substituted with 1 or 2 groups selected from R³; and

R³ is halo, (C₁-C₄)alkyl, or halo(C₁-C₄)alkyl;

the method comprising:

reacting a compound of Formula II,

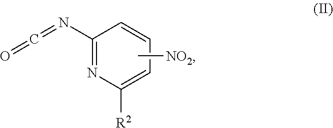

wherein R² is as described above for Formula I, with a compound having Formula III:

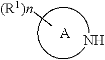

or a salt thereof, wherein Ring A, R¹ and n are as described above for Formula I, in the presence of a base to form the compound of Formula I.

In a second embodiment, the base used in the first embodiment is an organic base. Alternatively, as part of a second embodiment, the base used in the first embodiment is selected from the group consisting of pyridine, 4-dimethylamino pyridine, 2,3-lutidine, 2,6-lutidine, trimethylamine, diethylamine, dimethylamine, N,N-diisopropylethylamine, triethylamine, piperidine, 2,6-ditertbutylpyridine, 1,4-diazabicyclo[2.2.2]octane, and 1,8-diazabicyclo[5.4.0]undec-7-ene. In another alternative, the base used in the first embodiment is pyridine.

In a third embodiment, the compound of Formula II and the compound of Formula III are reacted in an organic solvent, wherein the variables and other conditions are as described in the first or second embodiment. Alternatively, as part of a third embodiment, the compound of Formula II and the compound of Formula III are reacted in an aprotic organic solvent, wherein the variables and other conditions are as described in the first or second embodiment. In another alternative, the compound of Formula II and the compound of Formula III are reacted in an organic solvent selected from the group consisting of hexane, benzene, toluene, anisole, 1,4-dioxane, chloroform, diethyl ether, dichloromethane, N-methylpyrrolidone, tetrahydrofuran, ethyl acetate, acetone, dimethylformamide, acetonitrile, and dimethylsulfoxide, or a combination thereof, wherein the variables and other conditions are as described in the first or second embodiment. In another alternative, the compound of Formula II and the compound of Formula III are reacted in a polar aprotic solvent, wherein the variables and other conditions are as described in the first or second embodiment. In another alternative, the compound of Formula II and the compound of Formula III are reacted in acetonitrile (ACN), wherein the variables and other conditions are as described in the first or second embodiment.

In a fourth embodiment, the compound of Formula III is a salt, wherein the variables and other conditions are as described in the first, second, or third embodiment. Alternatively, as part of a fourth embodiment, the compound of Formula III is an acid addition salt, wherein the variables and other conditions are as described in the first, second, or third embodiment. In another alternative, the compound of Formula III is a benzenesulfonic acid salt, a citric acid salt, a fumaric acid salt, a hydrochloric acid salt, a dihydrochloride salt, a malic acid salt, a methanesulfonic acid salt, a sulfuric acid salt, a tartaric acid salt, a trifluoroacetic acid salt, or a phosphoric acid salt, wherein the variables and other conditions are as described in the first, second, or third embodiment. In another alternative, the compound of Formula III is a hydrochloride salt, wherein the variables and other conditions are as described in the first, second, or third embodiment. In another alternative, the compound of Formula III is a dihydrochloride salt, wherein the variables and other conditions are as described in the first, second, or third embodiment.

In a fifth embodiment, the compound of Formula I, or a salt thereof, is reacted under reductive conditions to form a compound of Formula I′:

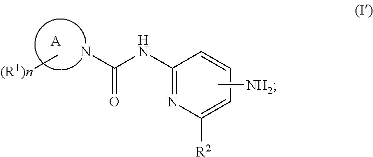

wherein the variables and other conditions are as described in the first, second, third, or fourth embodiment. Alternatively, as part of a fifth embodiment, the compound of Formula I is reacted under reductive conditions comprising catalytic hydrogenation (e.g., Raney nickel/H₂, Pd(C)/H₂, Pt(C)/H₂, Pd[Fe](C)/H₂, Pt[Fe](C)/H₂, Pt[V](C)/H₂, Pd[V](C)/H₂, Pd[Pt](C)/H₂), reaction with hydride donors (e.g., lithium aluminum hydride and boranes), reaction with metals in acidic media (e.g., iron or zinc with acetic acid), reaction with metals (e.g., tin(II) chloride, titanium(III) chloride, samarium, and zinc), reaction with acids (e.g., formic acid, ammonium formate, and hydroiodic acid) to form the compound of Formula I′, wherein the variables and other conditions are as described in the first, second, third, or fourth embodiment. In another alternative, the compound of Formula I, is reacted with formic acid in the presence of a metal catalyst (e.g., Pd(C)) to form the compound of Formula I′ or a salt thereof, wherein the variables and other conditions are as described in the first, second, third, or fourth embodiment.

In a sixth embodiment, the compound of Formula I, or a salt thereof, is reacted under reductive conditions in the presence of an organic base, wherein the variables and other conditions are as described in the first, second, third, fourth or fifth embodiment. Alternatively, as part of a sixth embodiment, the compound of Formula I, or a salt thereof, is reacted under reductive conditions in the presence of an organic base selected from the group consisting of methylamine, dimethylamine, diethylamine, trimethylamine, imidazole, N,N-disopropylethylamine, triethylamine, aniline, 4-methoxyaniline, and N,N-dimethylaniline, wherein the variables and other conditions are as described in the first, second, third, fourth or fifth embodiment. In another alternative, the compound of Formula I, or a salt thereof, is reacted under reductive conditions in the presence of an organic base, wherein the organic base is triethylamine; and wherein the variables and other conditions are as described in the first, second, third, fourth or fifth embodiment. In another alternative, the compound of Formula I, or a salt thereof, is reacted under reductive conditions in the presence of an organic base, wherein the organic base is trimethylamine; and wherein the variables and other conditions are as described in the first, second, third, fourth or fifth embodiment.

In a seventh embodiment, the compound of Formula I, or a salt thereof, is reacted under reductive conditions in the presence of an organic solvent, wherein the variables and other conditions are as described in the first, second, third, fourth, fifth or sixth embodiment. Alternatively, as part of a seventh embodiment, the compound of Formula I, or a salt thereof, is reacted under reductive conditions in the presence of an aprotic organic solvent, wherein the variables and other conditions are as described in the first, second, third, fourth, fifth or sixth embodiment. In another alternative, the compound of Formula I, or a salt thereof, is reacted under reductive conditions in the presence of an aprotic organic solvent selected from hexane, benzene, toluene, anisole, 1,4-dioxane, chloroform, diethyl ether, dichloromethane, N-methylpyrrolidone, tetrahydrofuran, ethyl acetate, acetone, dimethylformamide, acetonitrile, and dimethylsulfoxide, or a combination thereof, wherein the variables and other conditions are as described in the first, second, third, fourth, fifth or sixth embodiment. In another alternative, the compound of Formula I, or a salt thereof, is reacted under reductive conditions in the presence of an aprotic organic solvent, which is a combination of tetrahydrofuran and N-methylpyrrolidone, wherein the variables and other conditions are as described in the first, second, third, fourth, fifth or sixth embodiment. In another alternative, the compound of Formula I, or a salt thereof, is reacted under reductive conditions in the presence of an aprotic organic solvent, which is dimethylformamide, wherein the variables and other conditions are as described in the first, second, third, fourth, fifth or sixth embodiment.

In an eighth embodiment, the compound of Formula II is prepared by reacting a compound of Formula II′:

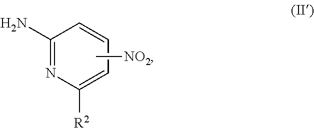

with an isocyanate former to form the compound of Formula II, wherein the variables and other conditions are as described in the first, second, third, fourth, fifth, sixth or seventh embodiment.

In a ninth embodiment, the isocyanate former used in the eighth embodiment is selected from the group consisting of phosgene, diphosgene, triphosgene, carbonyldiimidazole, and combinations of reagents such as CO₂/Mitsunobu zwitterions and (Boc)₂O/DMAP. Alternatively, as part of a ninth embodiment, the isocyanate former used in the eighth embodiment is triphosgene. In another alternative, the isocyanate former used in the eighth embodiment is phosgene.

In a tenth embodiment, the compound of Formula II is prepared by reacting the compound of Formula II′ with the isocyanate former in the presence of an organic base, wherein the variables and other conditions are as described in the first, second, third, fourth, fifth, sixth, seventh, eighth or ninth embodiment. Alternatively, as part of a tenth embodiment, the compound of Formula II is prepared by reacting the compound of Formula II′ with the isocyanate former in the presence of an organic base selected from the group consisting of pyridine, 4-dimethylamino pyridine, 2,3-lutidine, 2,6-lutidine, trimethylamine, diethylamine, dimethylamine, N,N-diisopropylethylamine, triethylamine, piperidine, 2,6-ditertbutylpyridine, 1,4-diazabicyclo[2.2.2]octane, and 1,8-diazabicyclo[5.4.0]undec-7-ene, wherein the variables and other conditions are as described in the first, second, third, fourth, fifth, sixth, seventh, eighth or ninth embodiment. In another alternative, the compound of Formula II is prepared by reacting the compound of Formula II′ with the isocyanate former in the presence of an organic base, which is pyridine, wherein the variables and other conditions are as described in the first, second, third, fourth, fifth, sixth, seventh, eighth or ninth embodiment.

In an eleventh embodiment, the compound of Formula II is prepared by reacting the compound of Formula II′ with the isocyanate former in the presence of an organic solvent, wherein the variables and other conditions are as described in the first, second, third, fourth, fifth, sixth, seventh, eighth, ninth or tenth embodiment. Alternatively, as part of an eleventh embodiment, the compound of Formula II is prepared by reacting the compound of Formula II′ with the isocyanate former in the presence of an aprotic organic solvent, wherein the variables and other conditions are as described in the first, second, third, fourth, fifth, sixth, seventh, eighth, ninth or tenth embodiment. In another alternative, the compound of Formula II is prepared by reacting the compound of Formula II′ with the isocyanate former in the presence of an aprotic organic solvent selected from the group consisting of hexane, benzene, toluene, anisole, 1,4-dioxane, chloroform, diethyl ether, dichloromethane, N-methylpyrrolidone, tetrahydrofuran, ethyl acetate, acetone, dimethylformamide, acetonitrile, and dimethylsulfoxide, or a combination thereof, wherein the variables and other conditions are as described in the first, second, third, fourth, fifth, sixth, seventh, eighth, ninth or tenth embodiment. In another alternative, the compound of Formula II is prepared by reacting the compound of Formula II′ with the isocyanate former in the presence of a polar aprotic organic solvent, wherein the variables and other conditions are as described in the first, second, third, fourth, fifth, sixth, seventh, eighth, ninth or tenth embodiment. In another alternative, the compound of Formula II is prepared by reacting the compound of Formula II′ with the isocyanate former in the presence of a polar aprotic organic solvent, which is acetonitrile (ACN), wherein the variables and other conditions are as described in the first, second, third, fourth, fifth, sixth, seventh, eighth, ninth or tenth embodiment.

In a twelfth embodiment, the compound of Formula I is of Formula Ia:

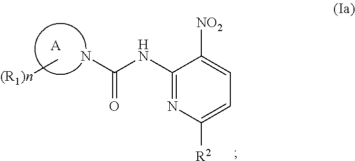

or a salt thereof; and the compound of Formula II disclosed herein above is of Formula IIa:

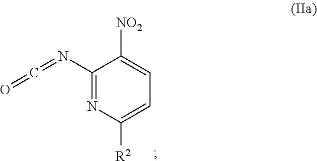

wherein the variables and conditions are as described in the first, second, third, fourth, fifth, sixth, seventh, eighth, ninth, tenth or eleventh embodiment.

In a thirteenth embodiment, R² in Formulae I, I′, II, II′, Ia, or IIa is phenyl or thienyl, each of which is optionally substituted with 1 or 2 groups selected from R³, wherein the other variables and conditions are as described in the first, second, third, fourth, fifth, sixth, seventh, eighth, ninth, tenth, eleventh or twelfth embodiment. Alternatively, as part of a thirteenth embodiment, R² in Formulae I, I′, II, II′, Ia, or IIa is phenyl optionally substituted with 1 or 2 groups selected from R³, wherein the other variables and conditions are as described in the first, second, third, fourth, fifth, sixth, seventh, eighth, ninth, tenth, eleventh or twelfth embodiment. Alternatively, as part of a thirteenth embodiment, R² in Formulae I, I′, II, II′, Ia, or IIa is thienyl optionally substituted with 1 or 2 groups selected from R³, wherein the other variables and conditions are as described in the first, second, third, fourth, fifth, sixth, seventh, eighth, ninth, tenth, eleventh or twelfth embodiment. Alternatively, as part of a twenty-seventh embodiment, R² in the compound of Formula I, Ia, I′, and I″ as described in the twenty-third and twenty-sixth embodiments is thienyl, wherein the variables and conditions are as described in the twenty-third, twenty-fourth, twenty-fifth, or twenty-sixth embodiment.

In a fourteenth embodiment, R³ in Formulae I, I′, II, II′, Ia, or IIa is halo, wherein the other variables and conditions are as described in the first, second, third, fourth, fifth, sixth, seventh, eighth, ninth, tenth, eleventh, twelfth, or thirteenth embodiment.

In a fifteenth embodiment, R² in Formulae I, I′, II, II′, Ia, or IIa is

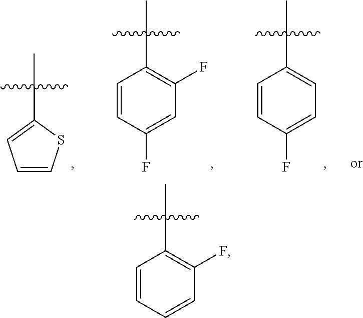 [SMILES: *c1cccs1 ; *c1ccc(F)cc1F ; *c1ccc(F)cc1 ; *c1ccccc1F]

wherein the other variables and conditions are as described in the first, second, third, fourth, fifth, sixth, seventh, eighth, ninth, tenth, eleventh, twelfth, thirteenth, or fourteenth embodiment. Alternatively, as part of a fifteenth embodiment, R² in Formulae I, I′, II, II′, Ia, or IIa is

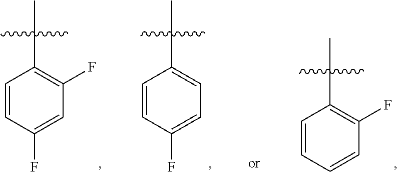 [SMILES: *c1ccc(F)cc1F ; *c1ccc(F)cc1 ; *c1ccccc1F]

wherein the other variables and conditions are as described in the first, second, third, fourth, fifth, sixth, seventh, eighth, ninth, tenth, eleventh, twelfth, thirteenth, or fourteenth embodiment. Alternatively, as part of a fifteenth embodiment, R² in Formulae I, I′ II, II′, Ia, or IIa is

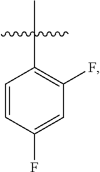 [SMILES: *c1ccc(F)cc1F]

wherein the other variables and conditions are as described in the first, second, third, fourth, fifth, sixth, seventh, eighth, ninth, tenth, eleventh, twelfth, thirteenth, or fourteenth embodiment. Alternatively, as part of a fifteenth embodiment, R² in Formulae I, I′, II, II′, Ia, or IIa is

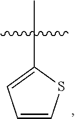 [SMILES: *c1cccs1]

wherein the other variables and conditions are as described in the first, second, third, fourth, fifth, sixth, seventh, eighth, ninth, tenth, eleventh, twelfth, thirteenth, or fourteenth embodiment.

In a sixteenth embodiment, ring A in Formulae I, I′, II, II′, Ia, or IIa is a monocyclic 4- to 6-membered heterocyclyl a 5,6-fused bicyclic heterocyclyl, or a 6,6-fused bicyclic heterocyclyl, wherein the other variables and conditions are as described in the first, second, third, fourth, fifth, sixth, seventh, eighth, ninth, tenth, eleventh, twelfth, thirteenth, fourteenth, or fifteenth embodiment. Alternatively, as part of a sixteenth embodiment, ring A in Formulae I, I′, II, II′, Ia, or IIa is

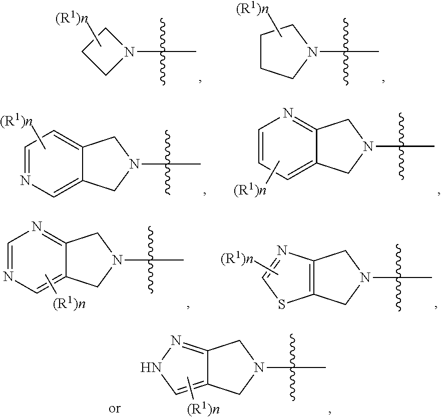

wherein the other variables and conditions are as described in the first, second, third, fourth, fifth, sixth, seventh, eighth, ninth, tenth, eleventh, twelfth, thirteenth, fourteenth, or fifteenth embodiment. In another alternative, ring A in Formulae I, I′, II, II′, Ia, or IIa is

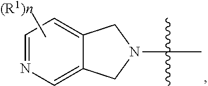

wherein the other variables and conditions are as described in the first, second, third, fourth, fifth, sixth, seventh, eighth, ninth, tenth, eleventh, twelfth, thirteenth, fourteenth, or fifteenth embodiment. In another alternative, ring A in Formulae I, I′, II, II′, Ia, or IIa is

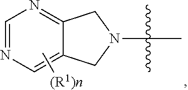

wherein the other variables and conditions are as described in the first, second, third, fourth, fifth, sixth, seventh, eighth, ninth, tenth, eleventh, twelfth, thirteenth, fourteenth, or fifteenth embodiment.

In a seventeenth embodiment, the variable n in Formulae I, I′, II, II′, Ia, or IIa is 1, wherein the other variables and conditions are as described in the first, second, third, fourth, fifth, sixth, seventh, eighth, ninth, tenth, eleventh, twelfth, thirteenth, fourteenth, fifteenth, or sixteenth embodiment.

In an eighteenth embodiment, R¹ in Formulae I, I′, II, II′, Ia, or IIa is pyrimidinyl or (C₁-C₄)alkyl optionally substituted with 1 to 3 groups selected from halo, pyrimidinyl, (C₁-C₄)alkoxy, or azetidinyl, wherein said azetidinyl and each instance of said pyrimidinyl is optionally substituted by 1 or 2 halo, wherein the other variables and conditions are as described in the first, second, third, fourth, fifth, sixth, seventh, eighth, ninth, tenth, eleventh, twelfth, thirteenth, fourteenth, fifteenth, sixteenth, or seventeenth embodiment. Alternatively, R¹ in Formulae I, I′, II, II′, Ia, or IIa is —CH₂OCH₃, wherein the other variables and conditions are as described in the first, second, third, fourth, fifth, sixth, seventh, eighth, ninth, tenth, eleventh, twelfth, thirteenth, fourteenth, fifteenth, sixteenth, or seventeenth embodiment. Alternatively, R¹ in Formulae I, I′, II, II′, Ia, or IIa is —CH₃, wherein the other variables and conditions are as described in the first, second, third, fourth, fifth, sixth, seventh, eighth, ninth, tenth, eleventh, twelfth, thirteenth, fourteenth, fifteenth, sixteenth, or seventeenth embodiment.

In a nineteenth embodiment, the compound of Formula I′ is of Formula IV:

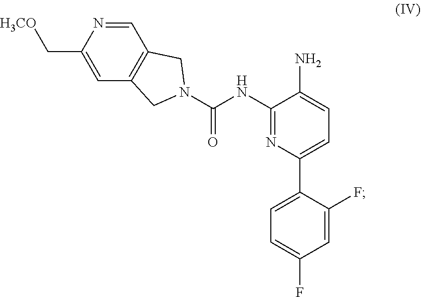 [SMILES: COCc1cc2c(cn1)CN(C(=O)Nc1nc(-c3ccc(F)cc3F)ccc1N)C2]

the compound of Formula I is of Formula V:

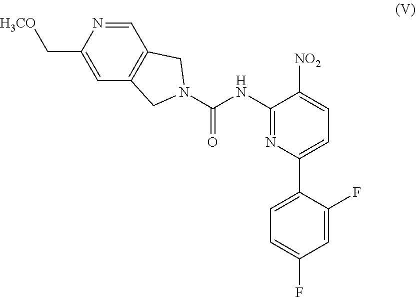 [SMILES: COCc1cc2c(cn1)CN(C(=O)Nc1nc(-c3ccc(F)cc3F)ccc1[N+](=O)[O-])C2]

or a salt thereof, the compound of Formula II is of Formula VI:

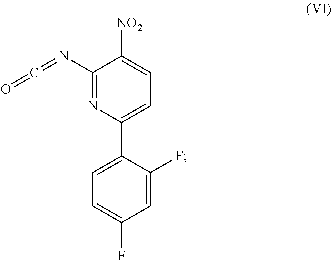 [SMILES: O=C=Nc1nc(-c2ccc(F)cc2F)ccc1[N+](=O)[O-]]

and the compound of Formula III is of Formula VII:

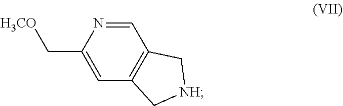 [SMILES: COCc1cc2c(cn1)CNC2]

or a salt thereof, wherein the variables and conditions are as described in the first, second, third, fourth, fifth, sixth, seventh, eighth, ninth, tenth or eleventh embodiment. Alternatively, as part of a nineteenth embodiment, the compound of Formula I′ is of Formula XI:

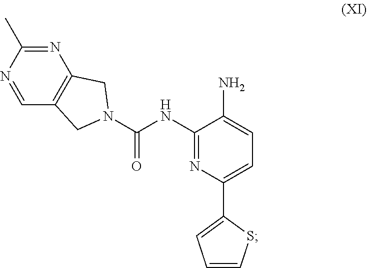 [SMILES: Cc1ncc2c(n1)CN(C(=O)Nc1nc(-c3cccs3)ccc1N)C2]

the compound of Formula I is of Formula XII:

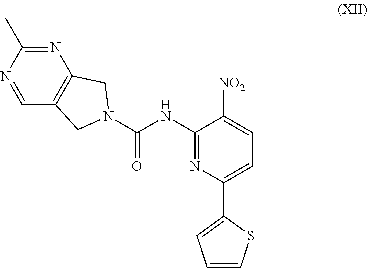 [SMILES: Cc1ncc2c(n1)CN(C(=O)Nc1nc(-c3cccs3)ccc1[N+](=O)[O-])C2]

or a salt thereof; the compound of Formula II is of Formula XIII:

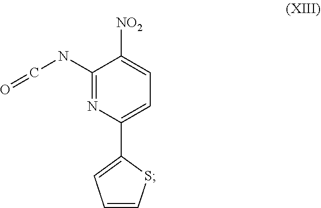 [SMILES: O=CNc1nc(-c2cccs2)ccc1[N+](=O)[O-]]

and the compound of Formula III is of Formula XIV:

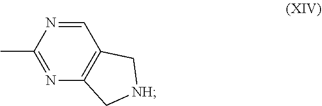

or a salt thereof, wherein the variables and conditions are as described in the first, second, third, fourth, fifth, sixth, seventh, eighth, ninth, tenth or eleventh embodiment.

In a twentieth embodiment, the compound of Formula IV is reacted under acidic conditions to form an acid addition salt. Alternatively, as part of a twentieth embodiment, the compound of Formula IV is reacted under acidic conditions to form a benzenesulfonic acid salt, a citric acid salt, a fumaric acid salt, a hydrochloric acid salt, a dihydrochloride salt, a malic acid salt, a methanesulfonic acid salt, a sulfuric acid salt, a tartaric acid salt, or a phosphoric acid salt.

In a twenty-first embodiment, the compound of Formula IV is reacted under acidic conditions to form a malate salt. Alternatively, as part of a twenty-first embodiment, the compound of Formula IV is reacted under acidic conditions to form a 1:1 molar ratio of malic acid to compound. In another alternative, the compound of Formula IV is reacted under acidic conditions to form an L-malate salt. In yet another alternative, the compound of Formula IV is reacted under acidic conditions to form a 1:1 molar ratio of L-malic acid to compound.

In a twenty-second embodiment, the compound of Formula IV is reacted under acidic conditions comprising malic acid and an aprotic solvent (e.g., N-methylpyrrolidone) in the presence of an anti-solvent (e.g., water, isopropyl alcohol or methyl-tert-butylether) to form an acid addition salt. Alternatively, as part of a twenty-second embodiment, the compound of Formula IV is reacted under acidic conditions comprising malic acid and an aprotic solvent (e.g., N-methylpyrrolidone) in the presence of an anti-solvent (e.g., water, isopropyl alcohol or methyl-tert-butylether) at variable temperatures (e.g., −5° C. to 25° C.).

Also provided herein, as part of a twenty-third embodiment, is a method of preparing a compound of Formula I′:

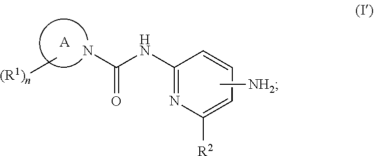

wherein:

ring A is a heterocyclyl;

R¹ is halo, (C₁-C₄)alkyl, halo(C₁-C₄)alkyl, (C₁-C₄)alkoxy, halo(C₁-C₄)alkoxy, or 4- to 6-membered monocyclic heteroaryl, where said (C₁-C₄)alkyl is optionally substituted with 1 or 3 groups selected from halo, hydroxyl, (C₁-C₄)alkoxy, —NH(C₁-C₄)alkyl, —N((C₁-C₄)alkyl)₂, and 4- to 6-membered monocyclic heteroaryl, wherein each instance of said 4- to 6-membered monocyclic heteroaryl is optionally substituted with 1 to 2 groups selected from halo, (C₁-C₄)alkoxy, (C₁-C₄)alkyl, and halo(C₁-C₄)alkyl;

n is 0, 1, or 2;

R² is phenyl or 5- to 6-membered monocyclic heteroaryl, each of which is optionally substituted with 1 or 2 groups selected from R³; and

R³ is halo, (C₁-C₄)alkyl, or halo(C₁-C₄)alkyl;

the method comprising:

reacting a compound having Formula I:

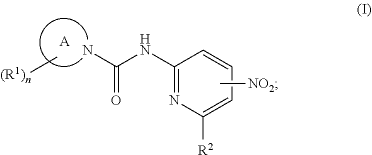

or a salt thereof, wherein the variables for Formula I are as defined above for Formula I′, with a metal catalyst (e.g., Raney nickel, Pd(C), Pt(C), Pd[Fe](C), Pt[Fe](C), Pt[V](C), Pd[V](C), or Pd[Pt](C)), an organic solvent, and optionally under an atmosphere of hydrogen and/or optionally with formic acid and an organic base to form the compound of Formula I′.

In a twenty-fourth embodiment, the organic base used in the formation of the compound of Formula I′ above is selected from the group consisting of methylamine, dimethylamine, diethylamine, trimethylamine, imidazole, N,N-disopropylethylamine, triethylamine, aniline, 4-methoxyaniline and N,N-dimethylaniline. Alternatively, as part of a twenty-fourth embodiment, the organic base used is triethylamine. Alternatively, as part of a twenty-fourth embodiment, the organic base used is trimethylamine.

In a twenty-fifth embodiment, the organic solvent used in the formation of the compound of Formula I′ as described in the twenty-third or twenty-fourth embodiment is aprotic. Alternatively, as part of a twenty-fifth embodiment, the organic solvent used in the formation of the compound of Formula I′ as described in the twenty-third or twenty-fourth embodiment is selected from the group consisting of hexane, benzene, toluene, anisole, 1,4-dioxane, chloroform, diethyl ether, dichloromethane, N-methylpyrrolidone, tetrahydrofuran, ethyl acetate, acetone, dimethylformamide, acetonitrile, and dimethylsulfoxide, or a combination thereof. In another alternative, the organic solvent used in the formation of the compound of Formula I′ as described in the twenty-third or twenty-fourth embodiment is a combination of tetrahydrofuran and N-methylpyrrolidone. In another alternative, the organic solvent used in the formation of the compound of Formula I′ as described in the twenty-third or twenty-fourth embodiment is dimethylformamide.

In a twenty-sixth embodiment, the compound of Formula I′ in the twenty-third embodiment is of Formula I′:

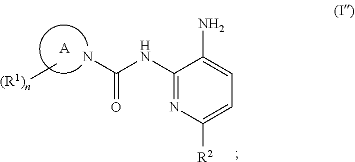

or a salt thereof, and the compound of Formula I in the twenty-third embodiment is of Formula Ia:

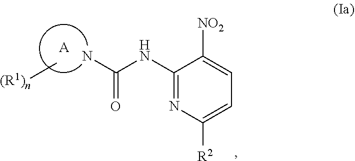

or a salt thereof, wherein the variables and conditions are as described in the twenty-third, twenty-fourth, or twenty-fifth embodiment.

In a twenty-seventh embodiment, R² in the compound of Formula I, Ia, I′, and I″ as described in the twenty-third and twenty-sixth embodiments is phenyl or thienyl, each of which is optionally substituted with 1 or 2 groups selected from R³, wherein the variables and conditions are as described in the twenty-third, twenty-fourth, twenty-fifth, or twenty-sixth embodiment. Alternatively, as part of a twenty-seventh embodiment, R² in the compound of Formula I, Ia, I′, and I″ as described in the twenty-third and twenty-sixth embodiments is phenyl optionally substituted with 1 or 2 groups selected from R³, wherein the variables and conditions are as described in the twenty-third, twenty-fourth, twenty-fifth, or twenty-sixth embodiment. Alternatively, as part of a twenty-seventh embodiment, R² in the compound of Formula I, Ia, I′, and I″ as described in the twenty-third and twenty-sixth embodiments is thienyl optionally substituted with 1 or 2 groups selected from R³, wherein the variables and conditions are as described in the twenty-third, twenty-fourth, twenty-fifth, or twenty-sixth embodiment. Alternatively, as part of a twenty-seventh embodiment, R² in the compound of Formula I, Ia, I′, and I″ as described in the twenty-third and twenty-sixth embodiments is thienyl, wherein the variables and conditions are as described in the twenty-third, twenty-fourth, twenty-fifth, or twenty-sixth embodiment.

In a twenty-eighth embodiment, R³ in the compound of Formula I, Ia, I′, and I″ as described in the twenty-third and twenty-sixth embodiments is halo, wherein the variables and conditions are as described in the twenty-third, twenty-fourth, twenty-fifth, twenty-sixth, or twenty-seventh embodiment.

In a twenty-ninth embodiment, R² in the compound of Formula I, Ia, I′, and I″ as described in the twenty-third and twenty-sixth embodiments is

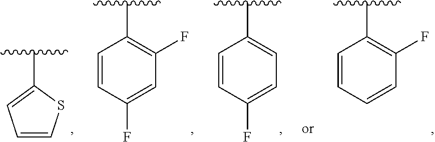

wherein the variables and conditions are as described in the twenty-third, twenty-fourth, twenty-fifth, twenty-sixth, twenty-seventh, or twenty-eighth embodiment. Alternatively, as part of a twenty-ninth embodiment, R² in the compound of Formula I, Ia, I′, and I″ as described in the twenty-third and twenty-sixth embodiments is

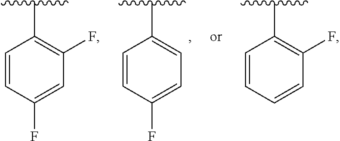

wherein the variables and conditions are as described in the twenty-third, twenty-fourth, twenty-fifth, twenty-sixth, twenty-seventh, or twenty-eighth embodiment. Alternatively, as part of a twenty-ninth embodiment, R² in the compound of Formula I, Ia, I′, and I″ as described in the twenty-third and twenty-sixth embodiments is

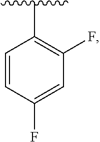 [SMILES: *c1ccc(F)cc1F]

wherein the variables and conditions are as described in the twenty-third, twenty-fourth, twenty-fifth, twenty-sixth, twenty-seventh, or twenty-eighth embodiment. Alternatively, as part of a twenty-ninth embodiment, R² in the compound of Formula I, Ia, I′, and I″ as described in the twenty-third and twenty-sixth embodiments is

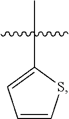 [SMILES: *c1cccs1]

wherein the variables and conditions are as described in the twenty-third, twenty-fourth, twenty-fifth, twenty-sixth, twenty-seventh, or twenty-eighth embodiment.

In a thirtieth embodiment, ring A in the compound of Formula I, Ia, I′, and I″ as described in the twenty-third and twenty-sixth embodiments is a monocyclic 4- to 6-membered heterocyclyl a 5,6-fused bicyclic heterocyclyl, or a 6,6-fused bicyclic heterocyclyl, wherein the variables and conditions are as described in the twenty-third, twenty-fourth, twenty-fifth, twenty-sixth, twenty-seventh, twenty-eighth, or twenty-ninth embodiment. Alternatively, as part of a thirtieth embodiment, ring A in the compound of Formula I, Ia, I′, and I″ as described in the twenty-third and twenty-sixth embodiments is

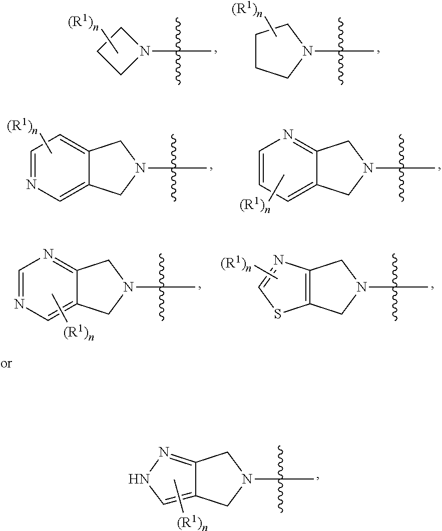

wherein the variables and conditions are as described in the twenty-third, twenty-fourth, twenty-fifth, twenty-sixth, twenty-seventh, twenty-eighth, or twenty-ninth embodiment. In another alternative, ring A in the compound of Formula I, Ia, I′, and I″ as described in the twenty-third and twenty-sixth embodiments is

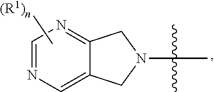

wherein the variables and conditions are as described in the twenty-third, twenty-fourth, twenty-fifth, twenty-sixth, twenty-seventh, twenty-eighth, or twenty-ninth embodiment. In another alternative, ring A in the compound of Formula I, Ia, I′, and I″ as described in the twenty-third and twenty-sixth embodiments is

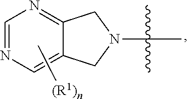

wherein the variables and conditions are as described in the twenty-third, twenty-fourth, twenty-fifth, twenty-sixth, twenty-seventh, twenty-eighth, or twenty-ninth embodiment.

In a thirty-first embodiment, the variable n in the compound of Formula I, Ia, I′, and I″ as described in the twenty-third and twenty-sixth embodiments is 1, wherein the variables and conditions are as described in the twenty-third, twenty-fourth, twenty-fifth, twenty-sixth, twenty-seventh, twenty-eighth, twenty-ninth, or thirtieth embodiment.

In a thirty-second embodiment, R¹ in the compound of Formula I, Ia, I′, and I″ as described in the twenty-third and twenty-sixth embodiments is pyrimidinyl or (C₁-C₄)alkyl optionally substituted with 1 to 3 groups selected from halo, pyrimidinyl, (C₁-C₄)alkoxy, or azetidinyl, wherein said azetidinyl and each instance of said pyrimidinyl is optionally substituted by 1 or 2 halo, wherein the variables and conditions are as described in the twenty-third, twenty-fourth, twenty-fifth, twenty-sixth, twenty-seventh, twenty-eighth, twenty-ninth, thirtieth, or thirty-first embodiment. Alternatively, as part of a thirty-second embodiment, R¹ in the compound of Formula I, Ia, I′, and I″ as described in the twenty-third and twenty-sixth embodiments is —CH₂OCH₃, wherein the variables and conditions are as described in the twenty-third, twenty-fourth, twenty-fifth, twenty-sixth, twenty-seventh, twenty-eighth, twenty-ninth, thirtieth, or thirty-first embodiment. Alternatively, as part of a thirty-second embodiment, R¹ in the compound of Formula I, Ia, I′, and I″ as described in the twenty-third and twenty-sixth embodiments is —CH₃, wherein the variables and conditions are as described in the twenty-third, twenty-fourth, twenty-fifth, twenty-sixth, twenty-seventh, twenty-eighth, twenty-ninth, thirtieth, or thirty-first embodiment.

In a thirty-third embodiment, the compound of Formula I′ in the twenty-third embodiment is of Formula IV:

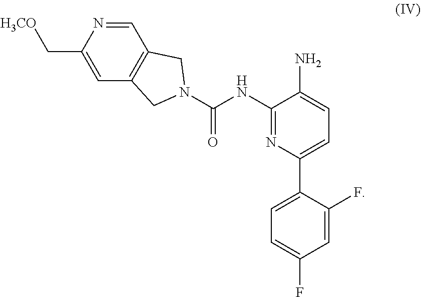 [SMILES: COCc1cc2c(cn1)CN(C(=O)Nc1nc(-c3ccc(F)cc3F)ccc1N)C2]

Alternatively, as part of a thirty-third embodiment, the compound of Formula I′ in the twenty-third embodiment is of Formula XI:

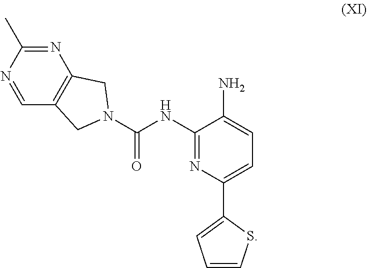 [SMILES: Cc1ncc2c(n1)CN(C(=O)Nc1nc(-c3cccs3)ccc1N)C2]

In a thirty-fourth embodiment, the compound of Formula IV in the thirty-third embodiment is reacted under acidic conditions to form an acid addition salt. Alternatively, as part of a thirty-fourth embodiment, the compound of Formula IV in the thirty-third embodiment is reacted under acidic conditions to form a benzenesulfonic acid salt, a citric acid salt, a fumaric acid salt, a hydrochloric acid salt, a dihydrochloride salt, a malic acid salt, a methanesulfonic acid salt, a sulfuric acid salt, a tartaric acid salt, or a phosphoric acid salt.

In a thirty-fifth embodiment, the compound of Formula IV in the thirty-third embodiment is reacted under acidic conditions to form a malate salt. Alternatively, as part of a thirty-fifth embodiment, the compound of Formula IV in the thirty-third embodiment is reacted under acidic conditions to form a 1:1 molar ratio of malic acid to compound. In another alternative, the compound of Formula IV in the thirty-third embodiment is reacted under acidic conditions to form an L-malate salt. In yet another alternative, the compound of Formula IV in the thirty-third embodiment is reacted under acidic conditions to form a 1:1 molar ratio of L-malic acid to compound.

In a thirty-sixth embodiment, the compound of Formula IV in the thirty-third embodiment is reacted under acidic conditions comprising malic acid and an aprotic solvent (e.g., N-methylpyrrolidone) in the presence of an anti-solvent (e.g., water, isopropyl alcohol or methyl-tert-butylether) to form an acid addition salt.

Also provided herein, as part of a thirty-seventh embodiment is an isocyanate compound having the Formula VIII:

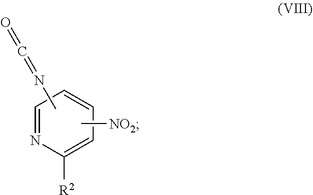

or a salt thereof, wherein R² is phenyl or 5- to 6-membered monocyclic heteroaryl, each of which is optionally substituted with 1 or 2 groups selected from R³; and R³ is halo, (C₁-C₄)alkyl, or halo(C₁-C₄)alkyl.

In a thirty-eighth embodiment, the compound of Formula VIII is of the Formula IX:

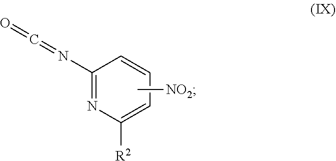

or a salt thereof, wherein R² is as defined above for Formula VIII.

In a thirty-ninth embodiment, the compound of Formula VIII is of the Formula X:

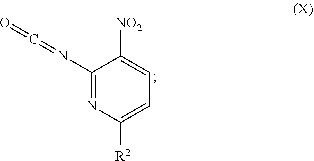

or a salt thereof, wherein R² is as defined above for Formula VIII.

In a fortieth embodiment, R² in the compounds of Formula VIII, IX, or X is phenyl or thienyl, each of which is optionally substituted with 1 or 2 groups selected from R³ (e.g., halo). Alternatively, R² in the compounds of Formula VIII, IX, or X is phenyl optionally substituted with 1 or 2 groups selected from R³ (e.g., halo). Alternatively, R² in the compounds of Formula VIII, IX, or X is thienyl optionally substituted with 1 or 2 groups selected from R³ (e.g., halo). Alternatively, R² in the compounds of Formula VIII, IX, or X is thienyl. In another alternative, R² in the compounds of Formula VIII, IX, or X is

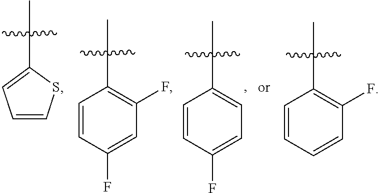

In another alternative, R² in the compounds of Formula VIII, IX, or X is

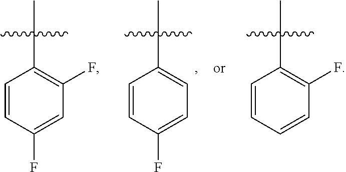

In yet another alternative, R² in the compounds of Formula VIII, IX, or X is

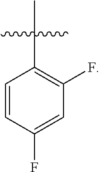 [SMILES: *c1ccc(F)cc1F]

In yet another alternative, R² in the compounds of Formula VIII, IX, or X is

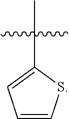 [SMILES: *c1cccs1]

Other compounds are described in the Exemplification section below and are included as part of the present disclosure. Free forms as well as salt forms of the compounds are included.

### EXEMPLIFICATION

N-(3-amino-6-(2,4-difluorophenyl)pyridin-2-yl)-6-(methoxymethyl)-1,3-dihydro-2H-pyrrolo[3,4-c]pyridine-2-carboxamide (Compound 1) was prepared via the procedure outlined below.

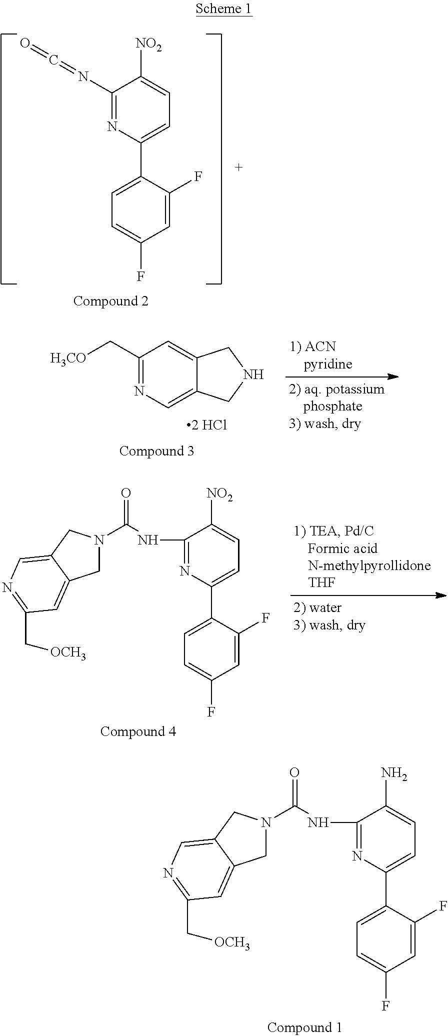

Synthesis of Compound 3

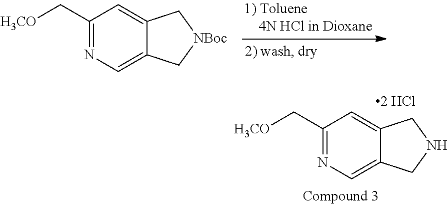 [SMILES: COCc1cc2c(cn1)CN(C(=O)OC(C)(C)C)C2 ; COCc1cc2c(cn1)CNC2]

A suspension of tert-butyl 6-(methoxymethyl)-1,3-dihydro-2H-pyrrolo[3,4-c]pyridine-2-carboxylate (7000 g) in toluene (12200 g) was stirred, with temperature maintained between 15-20° C., and then a solution of 4 M HCl in dioxane (58800 g) was added at such a rate to maintain the internal temperature of the reaction between 15-40° C. The addition may be paused at any time to account for exotherm. Upon completion of addition, the reaction was stirred for at least 12 h, maintaining internal temperature between 15-25° C. The reaction is then filtered to collect the solids, and the reaction flask rinsed with toluene (24400 g) to collect any residual material and filter. The solids are collected and dried in a vacuum oven between 45-60° C. for at least 12 h until the difference in weight of two consecutive weighings not less than 1 h apart is within 0.5 weight % (constant weight). Collected 6105 g (92% yield) of Compound 3 as a white solid.

Synthesis of Compound 2

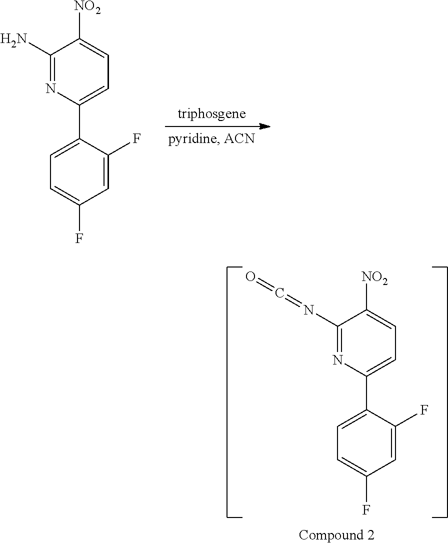

A reactor was charged with 6-(2,4-difluorophenyl)-3-nitropyridin-2-amine (4650 g), followed by triphosgene (2500 g) and acetonitrile (40900 g), and the reaction mixture was stirred at 0° C. Over the course of 2 h, pyridine (4570 g) was added to the reaction at such a rate as to maintain the internal temperature at 0° C. Upon completion of the pyridine addition, the reaction was allowed to warm to 20° C. and stirred for at least 12 h. This isocyanate reaction mixture as an intermediate was used in Step 3.

Synthesis of Compound 4

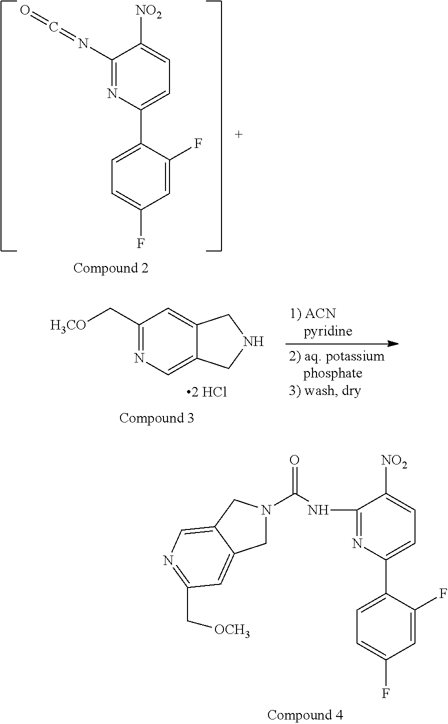

A sample of Compound 3 (5270 g) was taken up in acetonitrile (14600 g), and the mixture was treated with pyridine (4570 g) with stirring to create a transferable slurry. The slurry was then transferred to the isocyanate reaction mixture, maintaining the internal reaction temperature <35° C. The vessel which had contained Compound 3 slurry was rinsed with acetonitrile (3650 g×2), and each rinse was transferred to the reaction vessel. The reaction mixture was then stirred at 20° C. for at least 12 h, whereupon an aliquot was removed for monitoring reaction progress. The aliquot is treated with benzyl amine, and the amount of benzyl urea formed is used to monitor the amount of the isocyanate intermediate remaining. When the amount of benzyl urea formed is 5 area % benzyl urea, the reaction is quenched by addition of ethanol (3700 g), and the reaction mixture was stirred for at least 2 h. The reaction mixture was then treated with 0.5 M pH 7 potassium phosphate solution (˜45 L), resulting in precipitation of the desired product Compound 4. The reaction mixture is stirred for at least 12 h, maintaining the internal temperature at 20° C., and then the mixture was filtered to collect the solids. The reaction flask was rinsed with 0.5 M pH 7 potassium phosphate solution (˜45 L), filtered, and then rinsed again with 0.5 M pH 7 potassium phosphate solution (˜45 L). The filter cake was then sequentially rinsed with water (46600 g) and MTBE (20700 g×3), and vacuum maintained until the solids were dry enough to be transferred. The solids were then dried in a vacuum oven between 45-60° C. for at least 12 h. The material is dried until the difference in weight of two consecutive weighings not less than 1 h apart is within 0.5 weight % (constant weight). Collected 5781 g (71% yield from Compound 3) of Compound 4 as an off-white solid.

Synthesis of Compound 1

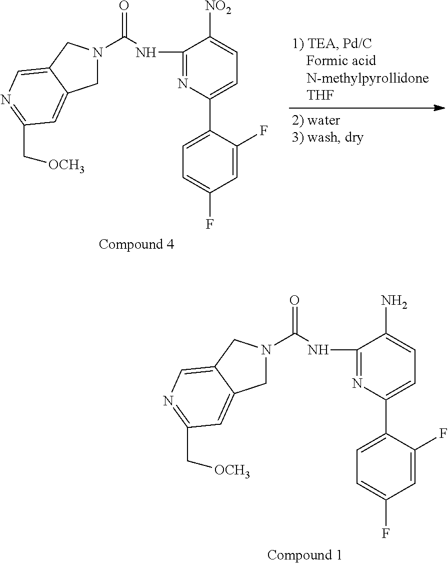 [SMILES: COCc1cc2c(cn1)CN(C(=O)Nc1nc(-c3ccc(F)cc3F)ccc1[N+](=O)[O-])C2 ; COCc1cc2c(cn1)CN(C(=O)Nc1nc(-c3ccc(F)cc3F)ccc1N)C2]

A solution of Compound 4 (5600 g) in NMP (28840 g) was broken into aliquots. A reactor was charged with 10% palladium on carbon (1680 g), followed by THE (24724 g), and triethylamine (5140 g). The mixture was stirred for 5 min, then 1/10^{th} of the solution of Compound 4 in NMP (3444 g) was added, with the internal temperature being maintained ≤35° C., and then formic acid (234 g) was added, with the internal temperature being maintained ≤35° C. An aliquot is removed to monitor the formation of hydroxylamine in the reaction mixture. After stirring ˜1 h, an additional 1/10^{th} of the solution of Compound 4 in NMP (3444 g) was added, with the internal temperature being maintained ≤35° C., and then formic acid (234 g) was added, with the internal temperature being maintained ≤35° C. After stirring ˜1 h, ⅕^{th} of the solution of Compound 4 in NMP (6888 g) was added, with the internal temperature being maintained ≤35° C., and then formic acid (468 g) was added with the internal temperature being maintained ≤35° C. After stirring ˜1 h, ⅕^{th} of the solution of Compound 4 in NMP (6888 g) was added, with the internal temperature being maintained ≤35° C., and then formic acid (468 g) was added with the internal temperature being maintained ≤35° C. After stirring ˜1 h, ⅕^{th} of the solution of Compound 4 in NMP (6888 g) was added, with the internal temperature being maintained ≤35° C., and then formic acid (468 g) was added with the internal temperature being maintained ≤35° C. After stirring ˜1 h, ⅕^{th} of the solution of Compound 4 in NMP (6888 g) was added, with the internal temperature being maintained ≤35° C., and then formic acid (468 g) was added with the internal temperature being maintained ≤35° C. When ≥90 area % compound 1 and ≤3 area % hydroxylamine remaining, reaction proceeds to workup. If reaction has stalled without meeting this criteria, a kicker charge of 10% palladium on carbon is added (in this case 560 g). When reaction progressed to completion, internal temperature was maintained at 15-20° C., Celite (840 g) was added to the reaction mixture as a filtration aid, and the mixture was filtered. To the filtrate was added NMP (8652 g) and then THF (7417 g). The mixture was stirred for 5 minutes, then was filtered and transferred to a mixing vessel. The prior vessel was rinsed with NMP (500 g), and then water (15680 g) was added to the mixing vessel to crystallize out the desired product. Seed crystals of the compound 1 (25 g) were added, and the mixture was stirred for at least 1 h, then additional water (49280 g) was added, and the mixture was stirred for 1-3 h.

The mixture was then filtered to collect the crystallized product, and the solids were washed with water (22400 g), and then isopropyl alcohol (17600 g). The solids were then dried in a vacuum oven overnight. Compound 1 was taken up in NMP (24952 g) and THF (21391 g) and treated with SiliaMetS Triamine (1454 g) Pd-scavenger, and the mixture was stirred, maintaining the internal temperature at 10° C. The mixture was stirred for 6 h at this temperature, and then was filtered. The filter cake was rinsed with a mixture of NMP (4990 g) and THE (4278 g), which was used to rinse the mixing vessel. To the filtrate was then added water (9700 g) to crystallize the product. The mixture was seeded with crystals of Compound 1, and then additional water (48500 g) was added, and the mixture was stirred for 1-3 h. The mixture was filtered to collect the crystallized product, and the solids were washed with isopropyl alcohol (15229 g), and dried under vacuum. The material was dried in a vacuum oven at ≤45° C. for at least 12 h, until a constant weight (difference in weight of two consecutive composite weighings are within 0.5 wt %). Isolated 3645 g of Compound 1 as the free base (70% yield).

Formation of Malate Salt of Compound 1

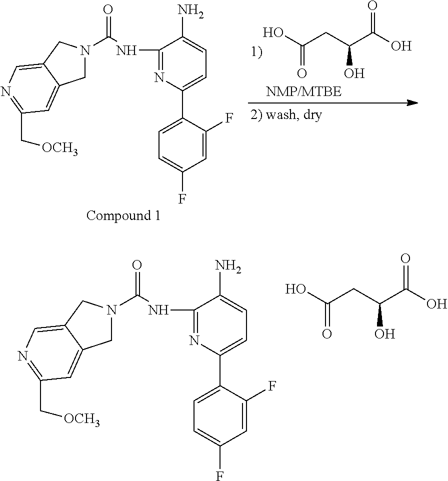 [SMILES: COCc1cc2c(cn1)CN(C(=O)Nc1nc(-c3ccc(F)cc3F)ccc1N)C2 ; O=C(O)C[C@H](O)C(=O)O ; COCc1cc2c(cn1)CN(C(=O)Nc1nc(-c3ccc(F)cc3F)ccc1N)C2 ; O=C(O)C[C@H](O)C(=O)O]

200 g of Compound 1 was dissolved with 49.5 g L-malic acid in 1400 mL NMP at 5° C. 10.1 g of malate Form A seed was suspended in 12.6 L MTBE at 5° C. The NMP solution was added to the MTBE suspension over 10 h at 5° C. and then stirred at 5° C. for 57.5 h. The suspension was filtered and washed with 3×250 mL MTBE. The wet cake was transferred to 3 L MTBE and the slurry was stirred at RT for 2 h and the suspension was filtered and washed with 2×250 mL MTBE. The wet cake was transferred to 3 L n-heptane and the slurry was stirred at RT for 2.5 h and the suspension was filtered and washed with 2×250 mL n-heptane. The cake was dried at RT under vacuum for 19 h and further dried at 40° C. under vacuum for 67 h (collected 193 g solid).

Alternatively, a 22 L flask was charged with L-malic acid (1273 g), followed by acetone (5570 g), and the mixture was stirred at RT. A separate reactor was charged with Compound 1 (3550 g), followed by anisole (42390 g) and then acetone (11120 g), and this slurry mixture was stirred at RT. The L-malic acid solution was added to the slurry of Compound 1 over the course of 1 h. The 22 L flask from the L-malic acid solution was then rinsed with acetone (395 g), which was added to the reactor containing the Compound 1/L-malic acid slurry. The mixture was stirred at RT for at least 12 h, then was filtered to collect the solids as Compound 1 malate salt. The solids were washed with heptane (2×9700 g), then were dried in a vacuum oven at ≤45° C. for at least 12 hours until reaching a constant weight (difference in weight of two consecutive composite weighings are within 0.5 wt %).

A 22 L flask was charged with Compound 1 L-malate salt (1800 g), followed by NMP (6490 g), and the solution was transferred to a 100 L reactor. To the initial 22 L reactor was added NMP (927 g) followed by isopropyl alcohol (707 g), and this mixture was transferred to the 100 L reactor with stirring. An additional portion of isopropyl alcohol (3113 g) was then added to the 100 L reactor, followed by seed crystals of Compound 1 malate Form A (5 g) as described below. Another portion of isopropyl alcohol (18817 g) was added to the 100 L reactor over the course of 3 h, and the mixture was stirred for at least 8 h. After removing an aliquot of the crystallized solids to test by XRPD and confirming the identity as Compound 1 malate Form A, the mixture was filtered to collect the crystallized solids. The filter cake was washed with isopropyl alcohol (7074 g), then was rinsed with two portions of heptane (18468 g, 6156 g). The solids were then dried in a vacuum oven (3 bar, ≤40° C.) for at least 12 h, until reaching a constant weight (difference in weight of two consecutive composite weighings are within 0.5 wt %). Collected 1308 g of Compound 1 malate Form A as a white solid (73% yield).

The seed crystals of Compound 1 malate Form A can be prepared by the following procedure. Starting from the free-base amorphous form as prepared following the procedure outline in Example 1 of U.S. Pat. No. 9,951,069, malic acid is added to any one of the following solvents: isopropyl alcohol:water 9:1, ethyl acetate, or ethanol.

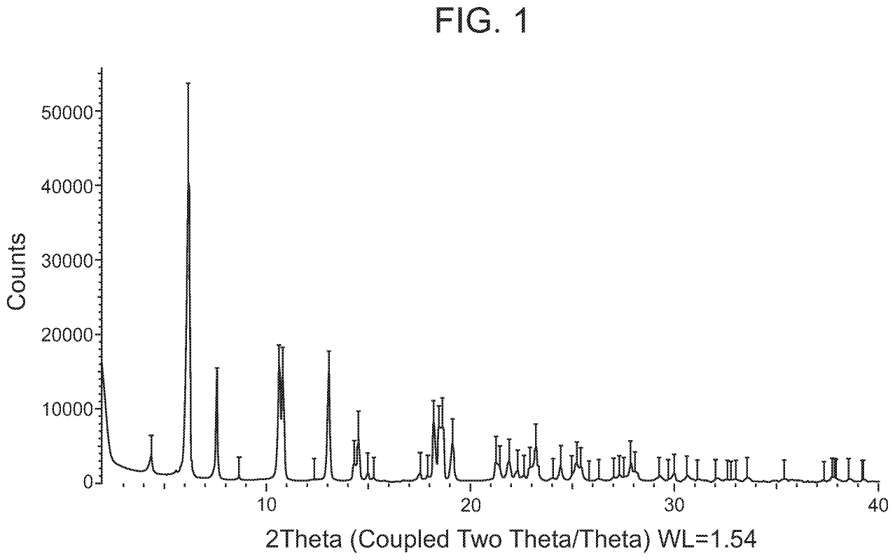

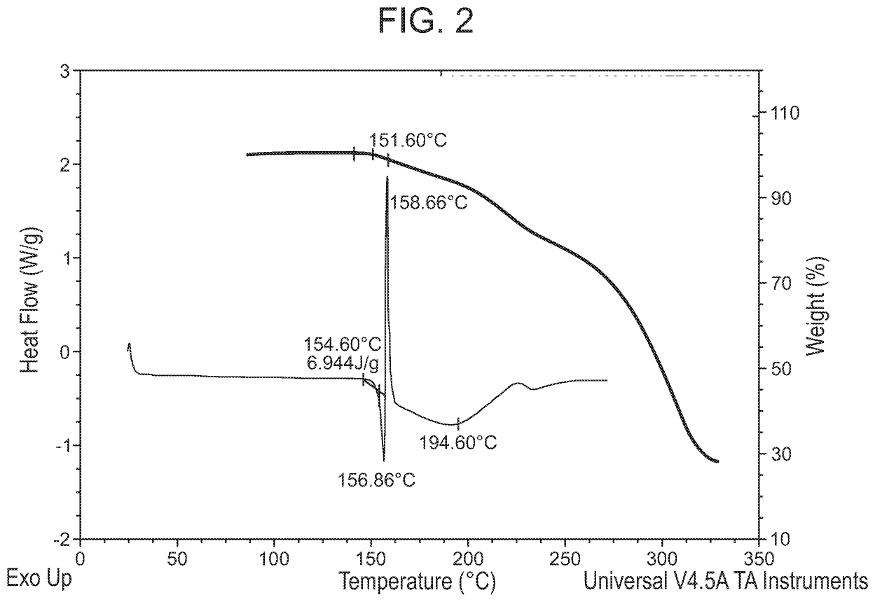

As a non-limiting example, a stock solution of the free-base amorphous form of Compound 1 is prepared and dispensed to a vial to give 20 mg of the free-base amorphous form. To the vial, 1.2 molar equivalents of malic acid is added, this left to stir overnight. Solvent is then evaporated to dryness and ethanol is added to each vial, the resulting slurry is left to stir for two days before samples are filtered. An exemplary XRPD, TGA, and DSC for Compound 1 malate Form A are shown in FIG. 1 and FIG. 2. Exemplary XRPD peaks of Compound 1 malate Form A are also provided in the table below. XRPD patterns/assignments recited herein are not to be construed as absolute and can vary e.g., by ±0.2 degrees. Similarly, temperature values for DSC peaks are not to be construed as absolute and can vary e.g., ±5 degrees or ±2 degrees.

Malate Form A

| 2Θ Angle | Rel. Intensity |
|---|---|
| 4.4 | 4.7 |
| 6.2 | 100 |
| 7.7 | 24.4 |
| 8.8 | 1.0 |
| 10.6 | 31.1 |
| 10.8 | 30.5 |
| 12.4 | 0.6 |
| 13.1 | 29.2 |
| 13.2 | 13.4 |
| 14.4 | 5.5 |
| 14.6 | 13.4 |
| 15.0 | 2.2 |
| 15.3 | 1.0 |
| 17.5 | 2.6 |
| 17.9 | 1.7 |
| 18.2 | 16.4 |
| 18.5 | 15.0 |
| 18.6 | 17.0 |
| 19.2 | 11.5 |
| 21.3 | 6.8 |
| 21.5 | 4.4 |
| 21.9 | 6.2 |
| 22.3 | 3.2 |
| 22.6 | 1.8 |
| 22.9 | 3.7 |
| 23.2 | 10.0 |
| 24.1 | 0.9 |
| 24.5 | 4.5 |
| 25.0 | 1.6 |
| 25.3 | 5.1 |
| 25.5 | 3.8 |
| 25.9 | 0.3 |
| 26.4 | 0.8 |
| 27.1 | 1.1 |
| 27.3 | 1.8 |
| 27.6 | 1.3 |
| 27.9 | 5.5 |
| 28.1 | 2.7 |
| 29.3 | 1.5 |
| 29.7 | 0.9 |
| 30.0 | 2.1 |
| 30.7 | 1.6 |
| 31.2 | 0.6 |
| 32.1 | 1.0 |
| 32.7 | 0.8 |
| 32.8 | 0.5 |
| 33.1 | 0.8 |
| 33.6 | 1.2 |
| 35.4 | 0.7 |
| 37.3 | 0.3 |
| 37.8 | 1.3 |
| 37.9 | 1.1 |
| 38.6 | 1.0 |
| 39.3 | 0.6 |

N-(3-amino-6-(thiophen-2-yl)pyridin-2-yl)-2-methyl-5,7-dihydro-6H-pyrrolo[3,4-d]pyrimidine-6-carboxamide (Compound 5) was prepared via the procedure outlined below.

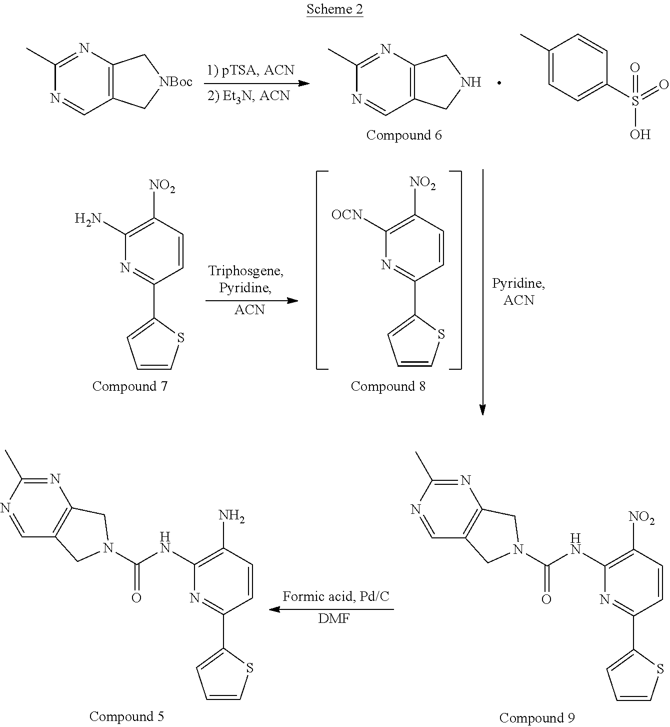

Synthesis of Compound 6

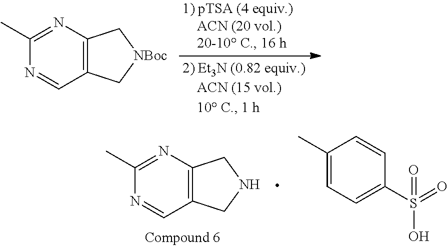

Dissolved tert-butyl 2-methyl-5,7-dihydro-6H-pyrrolo[3,4-d]pyrimidine-6-carboxylate [150.0 g, 96.0 wt %, 0.61 mol] in acetonitrile [5 vol, 750 ml] at 20° C. Dissolved p-toluenesulfonic acid [476.88 g, 2.48 mol, 4.0 equiv] in acetonitrile:2-methyl tetrahydrofuran [14:1 vol, 2100:150 ml] at 20° C. Charged p-toluenesulfonic acid solution to the tert-butyl 2-methyl-5,7-dihydro-6H-pyrrolo[3,4-d]pyrimidine-6-carboxylate solution dropwise over 15 minutes at 10° C. Aged mixture at 20° C. for 5 hours. Cooled to 10° C. for 16 hours. Filtered and washed cake with acetonitrile [3 vol, 450 ml]. Re-slurried cake in acetonitrile [15 vol, 2250 ml]. Charged triethylamine [71.54 ml, 0.51 mol, 0.82 equiv] to slurry over 15 minutes at 10° C. Aged mixture at 10° C. for 1 hour. Filtered and washed cake with acetonitrile [3 vol, 450 ml]. Dried solid at 25° C., under vacuum, for at least 16 h. Isolated 171 g (85% yield).

Synthesis of Compound 7

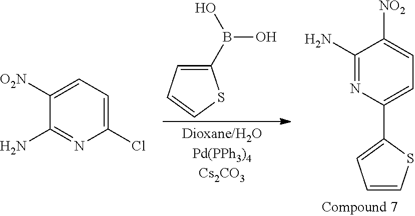 [SMILES: Nc1nc(Cl)ccc1[N+](=O)[O-] ; OB(O)c1cccs1 ; Nc1nc(-c2cccs2)ccc1[N+](=O)[O-]]

Charged H₂O (48.3 kg, 3.0 v/w) into a 1500 L reactor equipped with a temperature probe, overhead stirring at RT (15-25° C.) under N₂ protection. Charged Cs₂CO₃ (60.00 kg, 184.2 mol, 2.0 equiv) into the reactor at RT under N₂ protection. Charged dioxane (495.8 kg, 30 v/w) into the reactor at RT under N₂ protection. Charged 2-amino-6-chloro-3-nitropyridine (16.00 kg, 92.2 mol, 1.0 equiv) into the reactor at RT under N₂ protection over 20 min. Introduced N₂ gas into the reactor to remove O₂ at RT over 30 min. Charged Pd(PPh₃)₄ (3.41 kg, 2.95 mol, 0.032 equiv) into the reactor at RT under N₂ protection. Charged 2-thiopheneboronic acid (14.14 kg, 110.5 mol, 1.2 equiv) into the reactor at RT under N₂ protection over 10 min. Exchanged the atmosphere with N₂ by 3 times. Heated up the mixture to 65-70 degrees slowly over 2 h. Stirred the mixture at 70 degrees for another 1 h. Cooled down reaction mixture to 40° C. over 1 h. Charged ethyl acetate (288.6 kg, 18.0 w/w) into the reactor. Stirred at RT for 1 h. Filtered to remove undissolved solid through diatomite (16.0 kg, 1.0 w/w) over 12 h. Rinsed the cake with ethyl acetate (72.2 kg, 4.5 w/w). Washed the organics with 10% N-Acetyl-cysteine aqueous three times (52.8 kg, 3.3 w/w). Washed the organics with 10% NaCl aqueous (320.0 kg, 20 w/w). Charged Na₂SO₄ (32.00 kg, 2.0 w/w) into the resulting organic layer. Stirred over 30 min. Charged active carbon (11.20 kg, 0.7 w/w) into the reactor. Stirred over 1 h at RT. Filtered to remove solid. Rinsed the cake with ethyl acetate (72.2 kg, 4.5 w/w). Concentrated to remove the solvent at 50° C. under vacuum until the volume is about 64 L over 11 h. Charged isopropyl alcohol (62.8 kg, 3.9 w/w) into the reactor. Concentrated to remove the solvent at 50° C. under vacuum until the volume is about 64 L over 2.5 h. Charged isopropyl alcohol (62.9 kg, 3.9 w/w) into the reactor. Concentrated to remove the solvent at 50° C. under vacuum until the volume is about 64 L over 1 h. Charged isopropyl alcohol (62.9 kg, 3.9 w/w) into the reactor. Concentrated to remove the solvent at 50° C. under vacuum until the volume is about 64 L over 2 h. Heated to 70-80° C. over 1 h. Refluxed for 2 h. Cooled down to RT over 1 h. Filtered to collect the solid by centrifuge. Rinsed the cake with isopropyl alcohol (16.5 kg) and n-heptane (43.7 kg). Dried at 55° C. under vacuum over 24 h to afford compound as yellow solid (15.02 kg, 73.6% yield).

Synthesis of Compounds 8 and 9

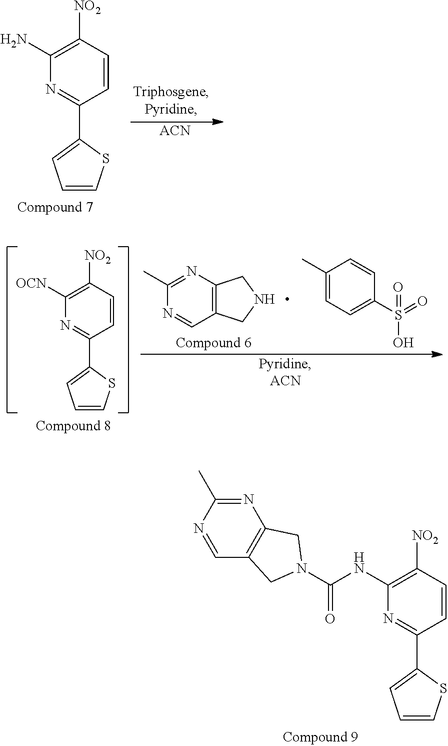

Charged 3-nitro-6-(thiophen-2-yl)pyridin-2-amine (50.0 g, 0.225 moles, 1.0 equiv.) and triphosgene (30.64 g, 0.101 mmol., 0.45 equiv.), slurried in acetonitrile (250 ml) and cooled to −10° C. Pyridine (53.9 g, 0.675 moles, 3.0 equiv.) was charged over 15 minutes, maintaining temperature below 0° C. Warmed to 20° C. and aged for 30 minutes, giving a yellow solution. In a separate vessel, 2-methyl-6,7-dihydro-5H-pyrrolo[3,4-d]pyrimidine 4-methylbenzenesulfonate, p-toluenesulfonic acid salt (89.7 g, 40.7 wt %, 0.27 moles, 1.2 equiv.) was slurried in acetonitrile (500 ml) and cooled to 0° C. Pyridine (53.9 g, 0.675 moles, 3.0 equiv.) was charged over 5 minutes, while temperature maintained at 0° C. The 2-isocyanato-3-nitro-6-(thiophen-2-yl)pyridine solution was then charged to the 2-methyl-6,7-dihydro-5H-pyrrolo[3,4-d]pyrimidine 4-methylbenzene slurry over 15 minutes, while maintaining the temperature below 10° C. The reaction was warmed to 20° C. and aged for 30 minutes. Water (1600 ml) was then charged over 30 minutes to crystallize the product and the resulting slurry aged at 20° C. for at least 2 hours. Filtered and washed the cake with 2:1 water:acetonitrile (450 ml) and then with water (3×150 ml). Dried the wet cake under vacuum at 50° C. for 18 h to yield an orange solid (84 g, 98% yield).

Synthesis of Compound 5

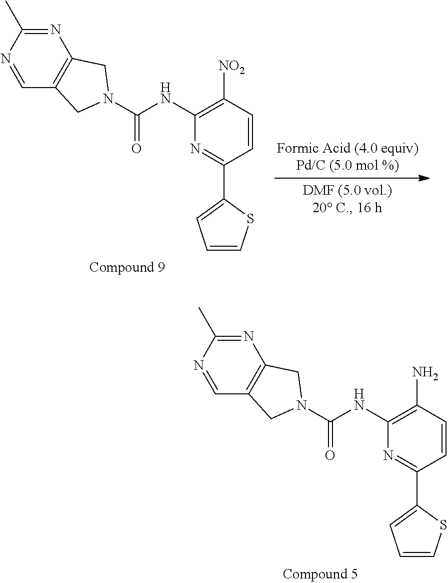 [SMILES: Cc1ncc2c(n1)CN(C(=O)Nc1nc(-c3cccs3)ccc1[N+](=O)[O-])C2 ; Cc1ncc2c(n1)CN(C(=O)Nc1nc(-c3cccs3)ccc1N)C2]

Charged 2-methyl-N-(3-nitro-6-(thiophen-2-yl)pyridin-2-yl)-5,7-dihydro-6H-pyrrolo[3,4-d]pyrimidine-6-carboxamide (30.0 g, 77.8 mmol., 1.0 equiv.), 5% Pd/C (23.0 g, 5 mol %, 67% water wet) and dimethylformamide (150 ml, 5.0 vol.) to a nitrogen purged reactor and stirred at 10° C. Charged formic acid (14.6 g, 311.0 mmol, 4.0 equiv.) while maintaining the temperature below 25° C. Charged triethylamine (3.9 g, 38.9 mmol, 0.5 equiv.) while maintaining the temperature below 25° C. Stirred at 20° C. until reaction was complete (8-16 hours). Filtered through SolkaFloc® with filter paper and washed the cake with dimethylformamide (90 ml, 3.0 vols). Recharged the solution to a clean reactor and charged Phosphonics™ SEA silica (24 g, 100 wt %). Filtered to remove resin. Recharged the solution to a clean reactor and charged methanol (990 ml, 33.0 vol.), then cooled to −20° C. The product is then ready to be filtered, washed, and/or dried.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of preparing a compound of Formula I, 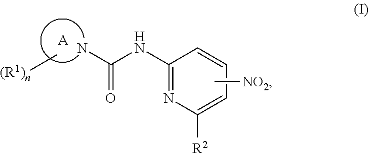
or a salt thereof, wherein:
ring A is a heterocyclyl;
R¹ is halo, (C₁-C₄)alkyl, halo(C₁-C₄)alkyl, (C₁-C₄)alkoxy, halo(C₁-C₄)alkoxy, or 4- to 6-membered monocyclic heteroaryl, where said (C₁-C₄)alkyl is optionally substituted with 1 to 3 groups selected from halo, hydroxyl, (C₁-C₄)alkoxy, —NH(C₁-C₄)alkyl, —N((C₁-C₄)alkyl)₂, and 4- to 6-membered monocyclic heteroaryl, wherein each instance of said 4- to 6-membered monocyclic heteroaryl is optionally substituted with 1 to 2 groups selected from halo, (C₁-C₄)alkoxy, (C₁-C₄)alkyl, and halo(C₁-C₄)alkyl;
n is 0, 1, or 2;
R² is phenyl or 5- to 6-membered monocyclic heteroaryl, each of which is optionally substituted with 1 or 2 groups selected from R³; and
R³ is halo, (C₁-C₄)alkyl, or halo(C₁-C₄)alkyl;
the method comprising:
reacting a compound of Formula II, 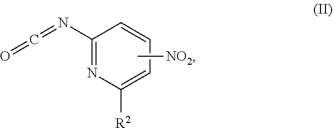
wherein R² is as described above for Formula I, with a compound having Formula III: 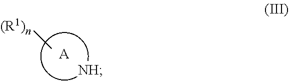
or a salt thereof, wherein R¹ and n are as described above for Formula I, in the presence of a base to form the compound of Formula I.

2-14. (canceled)

15. The method of claim 1, wherein the compound of Formula I, or a salt thereof, is reacted under reductive conditions to form a compound having Formula I′:

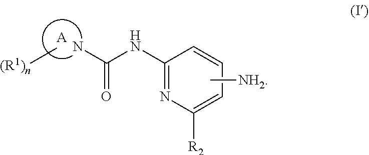

16-27. (canceled)

28. The method of claim 1, wherein the compound of Formula II is prepared by reacting a compound of Formula II′: 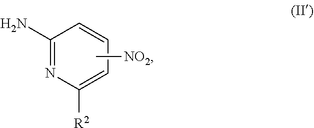
with an isocyanate former to form the compound of Formula II.

29-39. (canceled)

40. The method of claim 1, wherein the compound of Formula I is of Formula Ia: 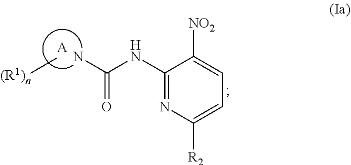
or a salt thereof;
and the compound of Formula II is of Formula IIa:

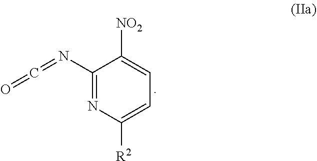

41-47. (canceled)

48. The method of claim 40, wherein R² is

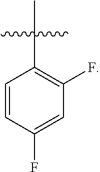 [SMILES: *c1ccc(F)cc1F]

49. The method of claim 40, wherein R² is

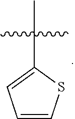 [SMILES: *c1cccs1]

50. (canceled)

51. (canceled)

52. The method of claim 40, wherein ring A is

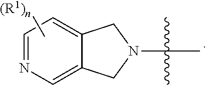

53. The method of claim 40, wherein ring A is

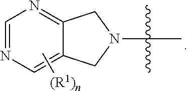

54. The method of claim 40, wherein n is 1.

55. (canceled)

56. The method of claim 40, wherein R¹ is —CH₂OCH₃.

57. The method of claim 40, wherein R¹ is —CH₃.

58. The method of claim 1, wherein the compound of Formula I′ is of Formula IV: 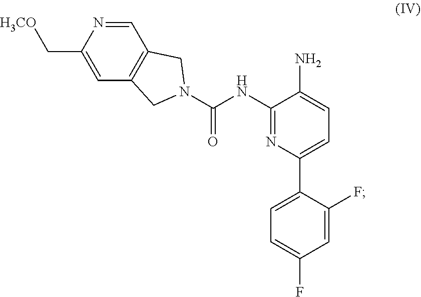 [SMILES: COCc1cc2c(cn1)CN(C(=O)Nc1nc(-c3ccc(F)cc3F)ccc1N)C2]
the compound of Formula I is of Formula V: 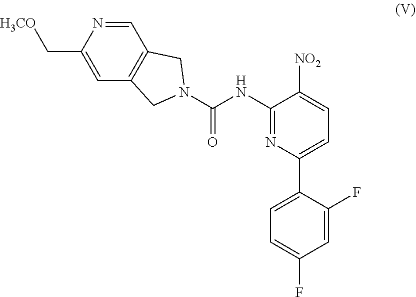 [SMILES: COCc1cc2c(cn1)CN(C(=O)Nc1nc(-c3ccc(F)cc3F)ccc1[N+](=O)[O-])C2]
or a salt thereof;
the compound of Formula II is of Formula VI: 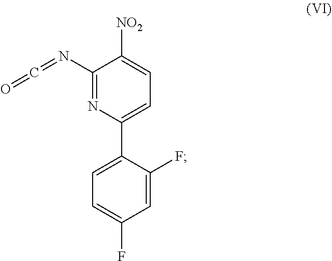 [SMILES: O=C=Nc1nc(-c2ccc(F)cc2F)ccc1[N+](=O)[O-]]
and
the compound of Formula III is of Formula VII: 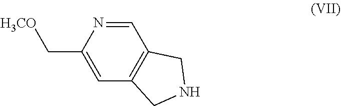 [SMILES: COCc1cc2c(cn1)CNC2]
or a salt thereof.

59. The method of claim 58, further comprising reacting the compound of Formula IV under acidic conditions to form an acid addition salt.

60. (canceled)

61. The method of claim 59, wherein the salt is a malate salt.

62. (canceled)

63. The method of claim 61, wherein the malate salt is an L-malate salt.

64-67. (canceled)

68. The method of claim 1, wherein the compound of Formula I′ is of Formula XI: 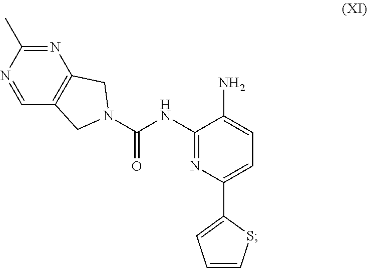 [SMILES: Cc1ncc2c(n1)CN(C(=O)Nc1nc(-c3cccs3)ccc1N)C2]
the compound of Formula I is of Formula XII: 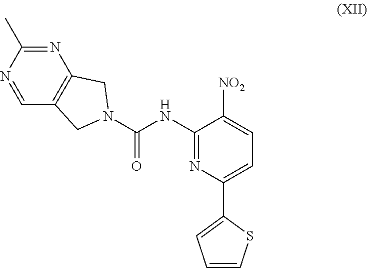 [SMILES: Cc1ncc2c(n1)CN(C(=O)Nc1nc(-c3cccs3)ccc1[N+](=O)[O-])C2]
or a salt thereof;
the compound of Formula II is of Formula XIII: 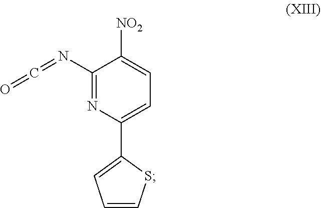 [SMILES: O=C=Nc1nc(-c2cccs2)ccc1[N+](=O)[O-]]
and
the compound of Formula III is of Formula XIV: 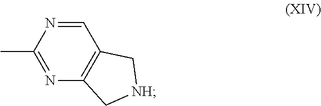
or a salt thereof.

69. A method of preparing a compound of Formula I′: 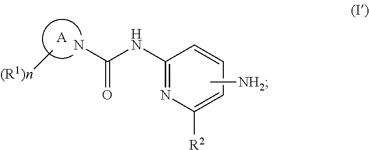
wherein:
ring A is a heterocyclyl;
R¹ is halo, (C₁-C₄)alkyl, halo(C₁-C₄)alkyl, (C₁-C₄)alkoxy, halo(C₁-C₄)alkoxy, or 4- to 6-membered monocyclic heteroaryl, where said (C₁-C₄)alkyl is optionally substituted with 1 or 3 groups selected from halo, hydroxyl, (C₁-C₄)alkoxy, —NH(C₁-C₄)alkyl, —N((C₁-C₄)alkyl)₂, and 4-to 6-membered monocyclic heteroaryl, wherein each instance of said 4- to 6-membered monocyclic heteroaryl is optionally substituted with 1 to 2 groups selected from halo, (C₁-C₄)alkoxy, (C₁-C₄)alkyl, and halo(C₁-C₄)alkyl;
n is 0, 1, or 2;
R² is phenyl or 5- to 6-membered monocyclic heteroaryl, each of which is optionally substituted with 1 or 2 groups selected from R³; and
R³ is halo, (C₁-C₄)alkyl, or halo(C₁-C₄)alkyl;
the method comprising:
reacting a compound having Formula I: 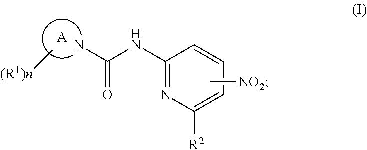
or a salt thereof, wherein the variables are as defined above for Formula I′, with formic acid, an organic base, a metal catalyst, and organic solvent to form the compound of Formula I′.

70-80. (canceled)

81. The method of claim 69, wherein the compound of Formula I′ is of Formula I″: 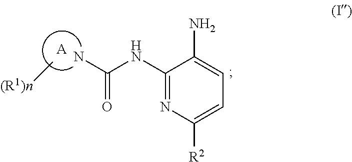
and the compound of Formula I is of Formula Ia: 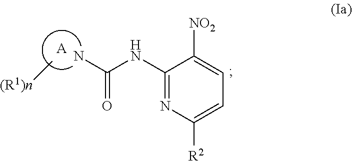
or a salt thereof.

82-88. (canceled)

89. The method of claim 81, wherein R² is

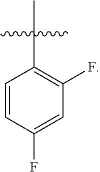 [SMILES: *c1ccc(F)cc1F]

90. The method of claim 81, wherein R² is

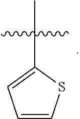 [SMILES: *c1cccs1]

91. (canceled)

92. (canceled)

93. The method of claim 81, wherein ring A is

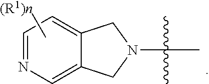

94. The method of claim 81, wherein ring A is

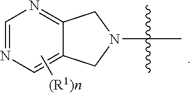

95. The method of claim 81, wherein n is 1.

96. (canceled)

97. The method of claim 81, wherein R¹ is —CH₂OCH₃.

98. The method of claim 81, wherein R¹ is —CH₃.

99. The method of claim 81, wherein the compound of Formula I′ is of Formula IV:

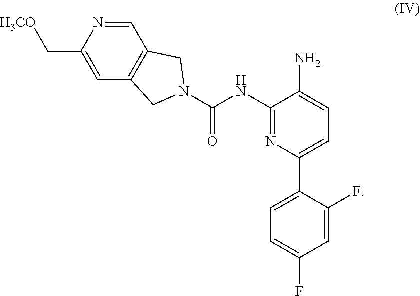 [SMILES: COCc1cc2c(cn1)CN(C(=O)Nc1nc(-c3ccc(F)cc3F)ccc1N)C2]

100. The method of claim 99, further comprising reacting the compound of Formula IV under acidic conditions to form an acid addition salt.

101. (canceled)

102. The method of claim 100, wherein the salt is a malate salt.

103. (canceled)

104. The method of claim 102, wherein the malate salt is an L-malate salt.

105-108. (canceled)

109. The method of claim 81, wherein the compound of Formula I′ is of Formula XI:

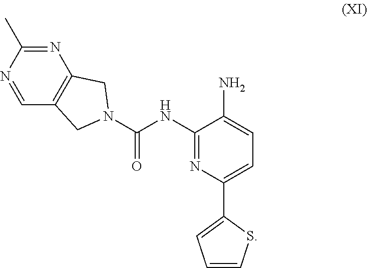 [SMILES: Cc1ncc2c(n1)CN(C(=O)Nc1nc(-c3cccs3)ccc1N)C2]

110. A compound having the Formula VIII: 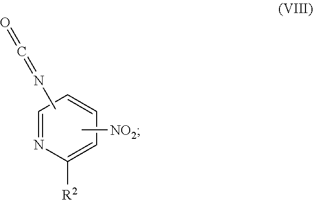
or a salt thereof, wherein
R² is phenyl or 5- to 6-membered monocyclic heteroaryl, each of which is optionally substituted with 1 or 2 groups selected from R³; and
R³ is halo, (C₁-C₄)alkyl, or halo(C₁-C₄)alkyl.

111. (canceled)

112. The compound of claim 110, wherein the compound is of the Formula X: 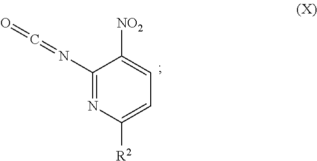
or a salt thereof.

113-119. (canceled)

120. The compound of claim 112, wherein R² is

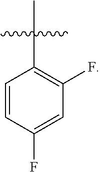 [SMILES: *c1ccc(F)cc1F]

121. The compound of claim 112, wherein R² is 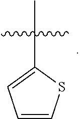 [SMILES: *c1cccs1]
